(19) United States
(12) Patent Application Publication
(10) Pub. No.: US 2021/0246208 A1
(43) Pub. Date: Aug. 12, 2021

(54) COMBINED INHIBITION OF PD-1/PD-L1, TGFB AND DNA-PK FOR THE TREATMENT OF CANCER

(71) Applicant: MERCK PATENT GMBH, Darmstadt (DE)

(72) Inventors: Yan LAN, Belmont, MA (US); Chunxiao XU, Belmont, MA (US)

(73) Assignee: MERCK PATENT GMBH, Darmstadt (DE)

(21) Appl. No.: 17/052,553

(22) PCT Filed: May 6, 2019
(86) PCT No.: PCT/EP2019/061558

§ 371 Date: Nov. 3, 2020

Related U.S. Application Data
(60) Provisional application No. 62/667,263, filed on May 4, 2018.

(51) Int. Cl. C07K 16/28; C07K 14/71; A61K 31/5377; A61P 35/00

(52) U.S. Cl. CPC C07K 16/2818; C07K 16/2827; A61K 2039/505; A61K 31/5377; A61P 35/00; C07K 14/71

(57) ABSTRACT

The present invention relates to combination therapies useful for the treatment of cancer. In particular, the invention relates to a therapeutic combination which comprises a PD-1 axis binding antagonist, a TGFβ inhibitor and a DNA-PK inhibitor, optionally together with one or more additional chemotherapeutic agents or radiotherapy. The therapeutic combination is particularly intended for use in treating a subject having a cancer that tests positive for PD-L1 expression.

### FIELD OF INVENTION

The present invention relates to combination therapies useful for the treatment of cancer. In particular, the invention relates to a therapeutic combination which inhibits PD-1/PD-L1, TGFβ and DNA-PK, optionally together with chemotherapy, radiotherapy or chemoradiotherapy. The therapeutic combination is particularly intended for use in treating a subject having a cancer that tests positive for PD-L1 expression.

### BACKGROUND OF THE INVENTION

Although radiation therapy is the standard of care to treat many different cancer types, treatment resistance remains a major concern. Mechanisms of resistance to radiation therapy are varied and complex, and include changes in DNA damage response pathways (DDR), modulation of immune cell functions, and increased levels of immunosuppressive cytokines like transforming growth factor beta (TGFβ). Strategies to combat resistance include combining radiation therapy with treatments that target these mechanisms.

DDR inhibitors are promising combination partners for radiation therapy. Radiation therapy kills cancer cells by damaging DNA, leading to activation of DDR pathways as cells attempt to repair the damage. Although DDR pathways are redundant in normal cells, one or more pathways is often lost during malignant progression, resulting in cancer cells relying more heavily on the remaining pathways and increasing the potential for genetic errors. This makes cancer cells uniquely vulnerable to treatment with DDR inhibitors. Since DNA double-strand breaks (DSBs) are considered the major cause of radiation-induced cell death, DDR inhibitors targeting DSB repair mechanisms like non-homologous end joining (NHEJ) may be particularly beneficial when used in combination with radiation therapy. Indeed, inhibitors of DNA-PK, a serine threonine kinase necessary for NHEJ, have demonstrated efficacy in sensitizing cancer cells to radiation therapy in preclinical models (ref). In the clinic, the DNA-PK inhibitor M3814 is being evaluated in combination with radiation therapy (clinicaltrials.gov identifier NCT02516813).

Treatments targeting immunosuppressive pathways such as TGFβ and programmed death ligand 1 (PD-L1)/programmed death 1 (PD-1) are also each being investigated alone or in combination with radiation therapy. The cytokine TGFβ has a physiological role in maintaining immunological self-tolerance, but in cancer, can promote tumor growth and immune evasion through effects on innate and adaptive immunity. The immune checkpoint mediated by PD-L1/PD-1 signaling dampens T cell activity and is exploited by cancer to suppress anti-tumor T cell responses. Both PD-L1 and TGF-β ligands are upregulated by radiation therapy and are thought to contribute to resistance.

US patent application publication number US 20150225483 A1, incorporated herein by reference, describes a bi-functional fusion protein that combines an anti-programmed death ligand 1 (PD-L1) antibody with the soluble extracellular domain of transforming growth factor beta receptor type II (TGFβRII) as a TGFβ neutralizing “Trap,” into a single molecule. Specifically, the protein is a heterotetramer, consisting of the two immunoglobulin light chains of anti-PD-L1, and two heavy chains comprising the heavy chain of anti-PD-L1 genetically fused via a flexible glycine-serine linker to the extracellular domain of the human TGFβRII (see FIG. 1). This anti-PD-L1/TGFβ Trap molecule is designed to target two major mechanisms of immunosuppression in the tumor microenvironment. US patent application publication number US 20150225483 A1 describes administration of the anti-PD-L1/TGFβ Trap molecule at doses based on the patient's weight. The international application PCT/US18/12604 describes body weight independent dosing regimens of the anti-PD-L1/TGFβ Trap molecule.

There remains a need to develop novel therapeutic options for the treatment of cancers. Furthermore, there is a need for therapies having greater efficacy than existing therapies. Preferred combination therapies of the present invention show greater efficacy than treatment with either therapeutic agent alone.

### SUMMARY OF THE INVENTION

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein. Embodiments within an aspect as described below can be combined with any other embodiments not inconsistent within the same aspect or a different aspect.

The present invention arises out of the discovery that a subject having a cancer can be treated with a combination of compounds which inhibit PD-1/PD-L1, TGFβ and DNA-PK. Treatment outcome can be further improved when the treatment with these compounds is combined with chemotherapy, radiotherapy or chemoradiotherapy. Thus, in a first aspect, the present invention provides a method comprising administering to the subject a PD-1 axis binding antagonist, a TGFβ axis binding antagonist and a DNA-PK inhibitor for treating a cancer in a subject in need thereof. Preferably, the PD-1 axis binding antagonist and the TGFβ inhibitor are fused. Also provided are methods of inhibiting tumor growth or progression in a subject who has malignant tumors. Also provided are methods of inhibiting metastasis of malignant cells in a subject. Also provided are methods of decreasing the risk of metastasis development and/or metastasis growth in a subject. Also provided are methods of inducing tumor regression in a subject who has malignant cells. The combination treatment results in an objective response, preferably a complete response or partial response in the subject. In some embodiments, the cancer is identified as PD-L1 positive cancerous disease.

Specific types of cancer to be treated according to the invention include, but are not limited to, cancer of the lung, head and neck, colon, neuroendocrine system, mesenchyme, breast, ovaries, pancreas, and histological subtypes thereof. In some embodiments, the cancer is selected from small-cell lung cancer (SCLC), non-small-cell lung cancer (NSCLC), squamous cell carcinoma of the head and neck (SCCHN), colorectal cancer (CRC), primary neuroendocrine tumors and sarcoma.

The PD-1 axis binding antagonist, TGFβ inhibitor and DNA-PK inhibitor, possibly in further combination with chemotherapy, radiotherapy or chemoradiotherapy, can be administered in a first-line, second-line or higher-line treatment of the cancer. In some embodiments, SCLC extensive disease (ED), NSCLC and SCCHN are selected for first-line treatment. In some embodiments, the cancer is resistant or became resistant to prior cancer therapy. The combination therapy of the invention can also be used in the treatment of a subject with the cancer who has been previously treated with one or more chemotherapies or underwent radiotherapy but failed with such previous treatment. The cancer for second-line or beyond treatment can be pre-treated relapsing metastatic NSCLC, unresectable locally advanced NSCLC, SCLC ED, pre-treated SCLC ED, SCLC unsuitable for systemic treatment, pre-treated relapsing or metastatic SCCHN, recurrent SCCHN eligible for re-irradiation, pre-treated microsatellite status instable low (MSI-L) or microsatellite status stable (MSS) metastatic colorectal cancer (mCRC), pre-treated subset of patients with mCRC (i.e., MSI-L or MSS), and unresectable or metastatic microsatellite instable high (MSI-H) or mismatch repair-deficient solid tumors progressing after prior treatment and which have no satisfactory alternative treatment options. In some embodiments, advanced or metastatic MSI-H or mismatch repair-deficient solid tumors progressing after prior treatment and which have no satisfactory alternative treatment options, are treated with the combination of the PD-1 axis binding antagonist, TGFβ inhibitor and DNA-PK inhibitor, possibly in further combination with chemotherapy, radiotherapy or chemoradiotherapy.

In a preferred embodiment, the subject to be treated is human.

In a preferred embodiment, the PD-1 axis binding antagonist is a biological molecule. Preferably, it is a polypeptide, more preferably an anti-PD-1 antibody or an anti-PD-L1 antibody. In some embodiments, the anti-PD-L1 antibody is used in the treatment of a human subject. In some embodiments, PD-L1 is human PD-L1.

In some embodiments, the anti-PD-L1 antibody comprises a heavy chain, which comprises three complementarity determining regions (CDRs) having amino acid sequences of SEQ ID NOs: 1, 2 and 3 corresponding to CDRH1, CDRH2 and CDRH3, respectively, and a light chain, which comprises three complementarity determining regions (CDRs) having amino acid sequences of SEQ ID NOs: 4, 5 and 6 corresponding to CDRL1, CDRL2 and CDRL3, respectively. The anti-PD-L1 antibody preferably comprises the heavy chain having amino acid sequences of SEQ ID NOs: 7 or 8 and the light chain having amino acid sequence of SEQ ID NO: 9. In some preferred embodiments, the anti-PD-L1 antibody is avelumab. In the most preferred embodiment, the anti-PD-L1 antibody is an anti-PD-L1 antibody fused to the extracellular domain of a TGFβ receptor II (TGFβR11) and comprises the heavy chain having amino acid sequence of SEQ ID NO: 10 and the light chain having amino acid sequence of SEQ ID NO: 9 (also referred to as “anti-PD-L1/TGFβ Trap” in the present disclosure).

In some embodiment, the anti-PD-L1 antibody is administered intravenously (e.g., as an intravenous infusion) or subcutaneously, preferably intravenously. More preferably, the anti-PD-L1 antibody is administered as an intravenous infusion. Most preferably, the inhibitor is administered for 50-80 minutes, highly preferably as a one-hour intravenous infusion. In some embodiment, the anti-PD-L1 antibody is administered at a dose of about 10 mg/kg body weight every other week (i.e., every two weeks, or “Q2W”). In some embodiments, the anti-PD-L1 antibody is administered at a fixed dosing regimen of 800 mg as a 1 hour IV infusion Q2W.

The TGFβ inhibitor may be a small molecule or a biological molecule, such as a polypeptide. In some embodiments, the TGFβ inhibitor is an anti-TGFβ antibody or a TGFβ receptor, such as the extracellular domain of human TGFβRII, or fragment thereof capable of binding TGFβ, acting as a TGFβ trap. In a preferred embodiment, the TGFβ inhibitor is fused to the PD-1 axis binding antagonist. More preferably, the TGFβ inhibitor is an extracellular domain of human TGFβRII, or fragment thereof capable of binding TGFβ, fused to an anti-PD-1 antibody or anti-PD-L1 antibody, such as the anti-PD-L1/TGFβ Trap described above.

In some aspects, the DNA-PK inhibitor is a small molecule. Preferably, it is (S)-[2-chloro-4-fluoro-5-(7-morpholin-4-yl-quinazolin-4-yl)-phenyl]-(6-methoxypyridazin-3-yl)-methanol (“Compound 1”) or a pharmaceutically acceptable salt thereof. In some embodiments, the DNA-PK inhibitor is administered orally. In some embodiments, the DNA-PK inhibitor is administered at a dose of about 1 to 800 mg once or twice daily (i.e., “QD” or “BID”). Preferably, the DNA-PK inhibitor is administered at a dose of about 100 mg QD, 200 mg QD, 150 mg BID, 200 mg BID, 300 mg BID or 400 mg BID, more preferably about 400 mg BID.

In a preferred embodiment, the recommended phase II dose for the DNA-PK inhibitor is 400 mg orally twice daily, and the recommended phase II dose for avelumab is 10 mg/kg IV every second week. In a preferred embodiment, the recommended phase II dose for the DNA-PK inhibitor is 400 mg twice daily as capsule, and the recommended phase II dose for avelumab is 800 mg Q2W.

In a preferred embodiment, the dose for the DNA-PK inhibitor is 400 mg orally twice daily (BID), and the dose for the anti-PD-L1/TGFβ Trap is 1200 mg IV every two weeks. In another preferred embodiment, the dose for the DNA-PK inhibitor is 400 mg orally twice daily (BID), and the dose for the anti-PD-L1/TGFβ Trap is 1800 mg IV every three weeks. In yet another preferred embodiment, the dose for the DNA-PK inhibitor is 400 mg orally twice daily (BID), and the dose for the anti-PD-L1/TGFβ Trap is 2400 mg IV every three weeks.

According to the invention, the PD-1 axis binding antagonist, the TGFβ inhibitor and the DNA-PK inhibitor can be fused in one or more molecules. Preferably, the PD-1 axis binding antagonist is fused to the TGFβ inhibitor, e.g., to form the anti-PD-L1/TGFβ Trap molecule described above.

In other embodiments, the PD-1 axis binding antagonist, the TGFβ inhibitor and the DNA-PK inhibitor are used in combination with chemotherapy (CT), radiotherapy (RT) or chemoradiotherapy (CRT). The chemotherapeutic agent can be etoposide, doxorubicin, topotecan, irinotecan, fluorouracil, gemcitabine, paclitaxel, a platin, an anthracycline, and a combination thereof. In a preferred embodiment, the chemotherapeutic agent can be doxorubicin. Preclinical studies showed an anti-tumor synergistic effect with DNA-PK inhibitors without adding a major toxicity.

In some embodiments, the etoposide is administered via intravenous infusion over about 1 hour. In some embodiments, the etoposide is administered on day 1 to 3 every three weeks (i.e., “D1-3 Q3W”) in an amount of about 100 mg/m². In some embodiments, the cisplatin is administered via intravenous infusion over about 1 hour. In some embodiments, the cisplatin is administered once every three weeks (i.e., “Q3W”) in an amount of about at 75 mg/m². In some embodiments, both etoposide and cisplatin are administered sequentially (at separate times) in either order or substantially simultaneously (at the same time).

In some embodiments, doxorubicin is administered every 21-28 days in an amount of 40 to 60 mg/m² IV. The dose and administration schedule could vary depending on the kind of tumor and the existing diseases and marrow reserves.

In some embodiments, the topotecan is administered on day 1 to 5 every three weeks (i.e., “D1-5 Q3W”).

In some embodiments, the anthracycline is administered until reaching a maximal life-long accumulative dose.

The radiotherapy can be a treatment given with electrons, photons, protons, alfa-emitters, other ions, radio-nucleotides, boron capture neutrons and combinations thereof. In some embodiments, the radiotherapy comprises about 35-70 Gy/20-35 fractions.

In a further aspect, the invention also relates to a method for advertising a PD-1 axis binding antagonist, a TGFβ inhibitor and a DNA-PK inhibitor in combination, preferably further in combination with chemotherapy, radiotherapy or chemoradiotherapy, comprising promoting, to a target audience, the use of the combination for treating a subject with a cancer, e.g., based on PD-L1 expression in samples, preferably tumor samples, taken from the subject. The PD-L1 expression can be determined by immunohistochemistry, e.g., using one or more primary anti-PD-L1 antibodies.

Provided herein is also a pharmaceutical composition comprising a PD-1 axis binding antagonist, a TGFβ inhibitor, a DNA-PK inhibitor and at least a pharmaceutically acceptable excipient or adjuvant, wherein the PD-1 axis binding antagonist and TGFβ inhibitor are preferably fused. The PD-1 axis binding antagonist, the TGFβ inhibitor and the DNA-PK inhibitor are provided in a single or separate unit dosage forms.

Also provided herein is a PD-1 axis binding antagonist, a TGFβ inhibitor and a DNA-PK inhibitor for the combined use in therapy, particularly for use in the treatment of cancer, wherein the administration of these compounds is preferably accompanied by chemotherapy, radiotherapy or chemoradiotherapy. Also provided herein is a PD-1 axis binding antagonist for use in therapy, particularly for use in the treatment of cancer, wherein the PD-1 axis binding antagonist is administered in combination with a TGFβ inhibitor and a DNA-PK inhibitor and, preferably, accompanied by chemotherapy, radiotherapy or chemoradiotherapy. Also provided herein is a TGFβ inhibitor for use in therapy, particularly for use in the treatment of cancer, wherein the TGFβ inhibitor is administered in combination with a PD-1 axis binding antagonist and a DNA-PK inhibitor and, preferably, accompanied by chemotherapy, radiotherapy or chemoradiotherapy. Also provided herein is a DNA-PK inhibitor for use in therapy, particularly for use in the treatment of cancer, wherein the DNA-PK inhibitor is administered in combination with a PD-1 axis binding antagonist and a TGFβ inhibitor and, preferably, accompanied by chemotherapy, radiotherapy or chemoradiotherapy. Also provided herein is a PD-1 axis binding antagonist fused to a TGFβ inhibitor for use in therapy, particularly for use in the treatment of cancer, wherein the PD-1 axis binding antagonist fused to the TGFβ inhibitor is administered in combination with a and a DNA-PK inhibitor and, preferably, accompanied by chemotherapy, radiotherapy or chemoradiotherapy.

Also provided is the use of a PD-1 axis binding antagonist, a TGFβ inhibitor and/or a DNA-PK inhibitor for the manufacture of a medicament, preferably for the treatment of cancer and wherein the administration of these compounds is preferably accompanied by chemotherapy, radiotherapy or chemoradiotherapy. Also provided is the use of a compound selected from the group consisting of PD-1 axis binding antagonist, a TGFβ inhibitor and a DNA-PK inhibitor for the manufacture of a medicament, preferably for the treatment of cancer, wherein the compound is administered in combination with the remaining compounds of this group of compounds and wherein the administration of these compounds is preferably accompanied by chemotherapy, radiotherapy or chemoradiotherapy. Also provided is the use of a PD-1 axis binding antagonist fused to a TGFβ inhibitor for the manufacture of a medicament, preferably for the treatment of cancer, wherein the PD-1 axis binding antagonist fused to the TGFβ inhibitor is administered in combination with a DNA-PK inhibitor and wherein the administration of these compounds is preferably accompanied by chemotherapy, radiotherapy or chemoradiotherapy.

Also provided is a method of treatment, preferably the treatment of cancer, comprising the administration of a PD-1 axis binding antagonist, a TGFβ inhibitor and a DNA-PK inhibitor, preferably in combination with chemotherapy, radiotherapy or chemoradiotherapy.

In a further aspect, the invention relates to a kit comprising a PD-1 axis binding antagonist and a package insert comprising instructions for using the PD-1 axis binding antagonist in combination with a TGFβ inhibitor and a DNA-PK inhibitor, preferably in further combination with chemotherapy, radiotherapy or chemoradiotherapy, to treat or delay progression of a cancer in a subject. In a further aspect, the invention relates to a kit comprising a TGFβ inhibitor and a package insert comprising instructions for using the TGFβ inhibitor in combination with a PD-1 axis binding antagonist and a DNA-PK inhibitor, preferably in further combination with chemotherapy, radiotherapy or chemoradiotherapy, to treat or delay progression of a cancer in a subject. In a further aspect, the invention relates to a kit comprising a PD-1 axis binding antagonist fused to a TGFβ inhibitor and a package insert comprising instructions for using the PD-1 axis binding antagonist fused to the TGFβ inhibitor in combination with a DNA-PK inhibitor, preferably in further combination with chemotherapy, radiotherapy or chemoradiotherapy, to treat or delay progression of a cancer in a subject. In a further aspect, the invention relates to a kit comprising a DNA-PK inhibitor and a package insert comprising instructions for using the DNA-PK inhibitor in combination with a TGFβ inhibitor and a PD-1 axis binding antagonist, preferably in further combination with chemotherapy, radiotherapy or chemoradiotherapy, to treat or delay progression of a cancer in a subject. In a further aspect, the invention relates to a kit comprising a PD-1 axis binding antagonist and a DNA-PK inhibitor and a package insert comprising instructions for using the PD-1 axis binding antagonist and the DNA-PK inhibitor in combination with a TGFβ inhibitor, preferably in further combination with chemotherapy, radiotherapy or chemoradiotherapy, to treat or delay progression of a cancer in a subject. In a further aspect, the invention relates to a kit comprising a TGFβ inhibitor and a DNA-PK inhibitor and a package insert comprising instructions for using the TGFβ inhibitor and the DNA-PK inhibitor in combination with a PD-1 axis binding antagonist, preferably in further combination with chemotherapy, radiotherapy or chemoradiotherapy, to treat or delay progression of a cancer in a subject. In a further aspect, the invention relates to a kit comprising a PD-1 axis binding antagonist, a TGFβ inhibitor and a DNA-PK inhibitor and a package insert comprising instructions for using the PD-1 axis binding antagonist, the TGFβ inhibitor and the DNA-PK inhibitor, preferably in further combination with chemotherapy, radiotherapy or chemoradiotherapy, to treat or delay progression of a cancer in a subject. The compounds of the kit may be comprised in one or more containers. In one embodiment, the kit comprises a first container, a second container and a package insert, wherein the first container comprises at least one dose of a medicament comprising a PD-1 axis binding antagonist fused to a TGFβ inhibitor, the second container comprises at least one dose of a medicament comprising a DNA-PK inhibitor, and the package insert comprises instructions for treating a subject for cancer using the medicaments, preferably in combination with chemotherapy, radiotherapy or chemoradiotherapy. The instructions can state that the medicaments are intended for use in treating a subject having a cancer that tests positive for PD-L1 expression by an immunohistochemical (IHC) assay.

In various embodiments, the PD-1 axis binding antagonist is fused to the TGFβ inhibitor and comprises the heavy chains and light chains of SEQ ID NO: 3 and SEQ ID NO: 1, respectively, of WO 2015/118175 and/or the DNA-PK inhibitor is (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof.

### BRIEF DESCRIPTION OF THE FIGURES

FIG. 1 shows the heavy chain sequence of avelumab and anti-PD-L1/TGFβ Trap. (A) SEQ ID NO: 7 represents the full length heavy chain sequence of avelumab. The CDRs having the amino acid sequences of SEQ ID NOs: 1, 2 and 3 are marked by underlining. (B) SEQ ID NO: 8 represents the heavy chain sequence of avelumab without the C-terminal lysine. The CDRs having the amino acid sequences of SEQ ID NOs: 1, 2 and 3 are marked by underlining. (C) SEQ ID NO: 10 represents the heavy chain sequence of anti-PD-L1/TGFβ Trap. The CDRs having the amino acid sequences of SEQ ID NOs: 1, 2 and 3 are marked by underlining.

FIG. 2 (SEQ ID NO: 9) shows the light chain sequence of avelumab and anti-PD-L1/TGFβ. The CDRs having the amino acid sequences of SEQ ID NOs: 4, 5 and 6 are marked by underlining.

FIG. 3 shows that Compound 1 (aka M3814) in combination with avelumab (without DNA damaging agent) increased the tumor growth inhibition and improved survival compared to single agent treatments in a syngeneic MC38 tumor model. M3814 was applied daily started from day 0; Avelumab was applied on days 3, 6 and 9.

FIG. 4 shows that a combination of radiotherapy, M3814 and avelumab resulted in a superior tumor growth control versus radiotherapy alone, radiotherapy and M3814, or radiotherapy and avelumab, in the syngeneic MC38 model.

FIG. 5 shows the anti-tumor effect of a combination of anti-PD-L1/TGFβ Trap (referred to as M7824), radiation therapy, and M3814 in the 4T1 model with concurrent or sequential dosing. BALB/c mice were inoculated intramuscularly (i.m.) with 0.5×10⁵ 4T1 cells (day −6) and treated (n=10 mice/group) with (A-C) isotype control (400 μg i.v.; day 0, 2, 4)+vehicle control (0.2 mL, orally [per os; p.o.], once daily [quaque die; q.d.], day 0-14), M7824 (492 μg i.v.; day 0, 2, 4), radiation (8 Gy, day 0-3), M3814 (150 mg/kg, p.o, q.d., day 0-14), M7824+RT, M7824+M3814, RT+M3814, or M7824+RT+M3814; or (D-F) isotype control (400 μg i.v.; day 4, 6, 8)+vehicle control (0.2 mL, (p.o.), (q.d.), day 0-14), M7824 (492 μg i.v.; day 4, 6, 8), radiation (8 Gy, day 0-3), M3814 (150 mg/kg, p.o, q.d., day 0-14), M7824+RT, M7824+M3814, RT+M3814, or M7824+RT+M3814. A-B, D-E, Tumor volumes were measured twice weekly and presented as (A, D) mean±SEM or (B, E) individual tumor volumes. P-values were calculated by two-way RM ANOVA with Tukey's post-test. C, F, For survival analysis, mice were sacrificed when tumor volumes reached ≈2000 mm³ and median survival times were calculated.

FIG. 6 shows the anti-tumor effect of a combination of anti-PD-L1/TGFβ Trap (referred to as M7824), radiation therapy, and M3814 in the GL261-Luc2 model. Albino C57BL/6 mice were inoculated orthotopically with 1×10⁶ GL261-Luc2 cells (day −7) via intracranial injections 1 mm anterior, 2 mm lateral (right), and 2 mm dorsal with respect to bregma. Mice were treated (n=8 mice/group) with isotype control (400 μg i.v.; day 0, 2, 4)+vehicle (0.2 mL p.o; days 0-14, radiation therapy (RT) (7.5 Gy, day 0), M7824 (492 μg i.v.; day 0, 2, 4)+RT, M3814 (150 mg/kg, p.o, q.d., day 0-14)+RT, or M7824+RT+M3814. Percent survival of mice was evaluated over the 91-day study. Mice were sacrificed when they were in a moribund state and median survival times were calculated.

FIG. 7 shows the anti-tumor effect of a combination of anti-PD-L1/TGFβ Trap (referred to as M7824), radiation therapy, and M3814 in the MC38 tumor model with concurrent dosing. C57BL/6 mice were inoculated i.m. with 0.25×10⁶ MC38 cells (day −6) and treated (n=10 mice/group) with isotype control (133 μg i.v.; day 0)+vehicle control (0.2 mL p.o., q.d., day 0-14), M7824 (164 μg i.v.; day 0), radiation (3.6 Gy, day 0-3), M3814 (50 mg/kg, p.o, q.d., day 0-14), M7824+RT, M7824+M3814, RT+M3814, or M7824+RT+M3814. A-B, Tumor volumes were measured twice weekly and presented as (A) mean±SEM or (B) individual tumor volumes. P-values were calculated by two-way RM ANOVA with Tukey's post-test. C, For survival analysis, mice were sacrificed when tumor volumes reached ≈2000 mm³ and median survival times were calculated.

FIG. 8 shows the anti-tumor effect of a combination of anti-PD-L1/TGFβ Trap (referred to as M7824), radiation therapy, and M3814 in the MC38 model. C57BL/6 mice were inoculated i.m. with 0.25×10⁶ MC38 cells in the right thigh (primary tumor) and s.c. with 1×10⁶ MC38 cells in the left flank (secondary tumor) (day −7). Mice (n=6 mice/group) were treated (day 0) with isotype control (133 μg i.v.; day 0)+vehicle control (0.2 mL p.o., q.d., days 0-14), M7824 (164 μg i.v. day 0)+vehicle, RT (3.6 Gy, day 0-3)+vehicle+isotype controls, M3814 (50 mg/kg p.o., q.d., day 0-14)+isotype control, M7824+M3814, M7824+RT, M3814+RT, or M7824+RT+M3814. Tumor volumes for the primary tumors (A) and secondary tumors (B) were measured twice weekly and presented as mean±SEM. P-values were calculated by two-way RM ANOVA with Tukey's post-test.

FIG. 9 shows the abscopal effect potentiated by the combination of anti-PD-L1/TGFβ Trap (referred to as M7824), radiation therapy, and M3814 in the 4T1 model. BALB/c mice were inoculated in the mammary fat pad with 0.5×10⁶ 4T1-Luc2-1A4 cells (day −9) and treated (n=8 mice/group) with isotype control (400 μg i.v.; day 0, 2, 4)+vehicle control (0.2 mL p.o., day 0-15), M7824 (492 μg i.v.; day 0, 2, 4), radiation (10 Gy, day 0), M7824+RT, RT+M3814 (150 mg/kg, p.o., day 0-15), or M7824+RT+M3814. Bioluminescence imaging (BLI) of the luciferase-expressing tumor cells was performed after systemic injection of D-luciferin to enable a noninvasive determination of site-localized tumor burden. (A) In vivo BLI images were acquired on Days 9, 14 and 21 post treatment start. Mean is shown as line. (B) Ex vivo BLI (photons/sec) of the lungs at Day 23 is plotted. P-values were calculated with a Mann-Whitney test. *P≤0.05, **P≤0.01, and ***P≤0.001 denote a significant difference relative to triple combination.

FIG. 10 shows the percentage of CD8⁺ cells in tumors treated with anti-PD-L1/TGFβ Trap (referred to as M7824), radiation therapy, and M3814 in the 4T1 model. BALB/c mice were inoculated i.m. with 0.5×10⁵ 4 T1 cells (day −7) and treated (n=10 mice/group) with isotype control (400 μg i.v.; day 0, 2, 4)+vehicle control (0.2 mL p.o., day 0-15), M7824 (492 μg i.v.; day 0, 2, 4), radiation (8 Gy, days 0-3), M3814 (150 mg/kg, days 0-10), M7824+RT, M7824+M3814, RT+M3814, or M7824+RT+M3814. Tumor tissues were harvested at Day 10 and stained for murine CD8a. (A) Representative images of anti-CD8a immunohistochemistry (IHC) of tumors (n=10 mice/group) and (B) percentages of CD8⁺ cells are shown. Scale bars, 100 μm.

FIG. 11 shows gene expression changes from tumors treated with anti-PD-L1/TGFβ Trap (referred to as M7824), radiation therapy, and M3814 in the 4T1 model. BALB/c mice were inoculated i.m. with 0.5×10⁵ 4T1 cells (day −6) and treated (n=10 mice/group) with isotype control (400 μg i.v.; day 0, 2, 4)+vehicle control (0.2 mL p.o., day 0-6), M7824 (492 μg i.v.; day 0, 2, 4), radiation (8 Gy, days 0-3), M3814 (150 mg/kg, days 0-6), M7824+RT, M7824+M3814, RT+M3814, or M7824+RT+M3814. Tumor tissues were harvested at Day 6 for RNAseq analysis. Gene expression signatures associated with (A) EMT, (B) fibrosis, and (C) VEGF pathway signatures are presented as box plots. Signature scores are defined as the mean log₂ (fold change) among all genes in the signature.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

“About” when used to modify a numerically defined parameter (e.g., the dose of a PD-1 axis binding antagonist, a TGFβ inhibitor or DNA-PK inhibitor, or the length of treatment time with a combination therapy described herein) means that the parameter may vary by as much as 10% below or above the stated numerical value for that parameter. For example, a dose of about 10 mg/kg may vary between 9 mg/kg and 11 mg/kg.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates, an antibody fused to a cytokine or an antibody fused to a cytokine receptor), any other modified configuration of the immunoglobulin molecule that comprises an antigen recognition site, antibody compositions with poly-epitopic specificity, and multi-specific antibodies (e.g., bispecific antibodies).

“Antigen-binding fragment” of an antibody or “antibody fragment” comprises a portion of an intact antibody, which is still capable of antigen binding and/or the variable region of the intact antibody. Antigen-binding fragments include, for example, Fab, Fab′, F(ab′)₂, Fd, and Fv fragments, domain antibodies (dAbs, e.g., shark and camelid antibodies), fragments including complementarity determining regions (CDRs), single chain variable fragment antibodies (scFv), single-chain antibody molecules, multi-specific antibodies formed from antibody fragments, maxibodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991).

“Anti-PD-L1 antibody” or “anti-PD-1 antibody” means an antibody, or an antigen-binding fragment thereof, that blocks binding of PD-L1 expressed on a cancer cell to PD-1. In any of the treatment methods, medicaments and uses of the present invention in which a human subject is being treated, the anti-PD-L1 antibody specifically binds to human PD-L1 and blocks binding of human PD-L1 to human PD-1. In any of the treatment methods, medicaments and uses of the present invention in which a human subject is being treated, the anti-PD-1 antibody specifically binds to human PD-1 and blocks binding of human PD-L1 to human PD-1. The antibody may be a monoclonal antibody, human antibody, humanized antibody or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments. Examples of monoclonal antibodies that bind to human PD-L1, and useful in the treatment method, medicaments and uses of the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 monoclonal antibodies useful as the PD-L1 antibody in the treatment method, medicaments and uses of the present invention include, for example without limitation, an antibody which comprises the heavy chains and light chains of SEQ ID NO: 3 and SEQ ID NO: 1, respectively, of WO 2015/118175, avelumab (MSB0010718C), nivolumab (BMS-936558), MPDL3280A (an IgG1-engineered, anti-PD-L1 antibody), BMS-936559 (a fully human, anti-PD-L1, IgG4 monoclonal antibody), MED14736 (an engineered IgG1 kappa monoclonal antibody with triple mutations in the Fc domain to remove antibody-dependent, cell-mediated cytotoxic activity), and an antibody which comprises the heavy chain and light chain variable regions of SEQ ID NO:24 and SEQ ID NO:21, respectively, of WO 2013/019906.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy. E.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

“Blood” refers to all components of blood circulating in a subject including, but not limited to, red blood cells, white blood cells, plasma, clotting factors, small proteins, platelets and/or cryoprecipitate. This is typically the type of blood which is donated when a human patient gives blood. Plasma is known in the art as the yellow liquid component of blood, in which the blood cells in whole blood are typically suspended. It makes up about 55% of the total blood volume. Blood plasma can be prepared by spinning a tube of fresh blood containing an anti-coagulant in a centrifuge until the blood cells fall to the bottom of the tube. The blood plasma is then poured or drawn off. Blood plasma has a density of approximately 1025 kg/m³ or 1.025 kg/l.

“Cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Chemotherapy” is a therapy involving a chemotherapeutic agent, which is a chemical compound useful in the treatment of cancer. Examples of chemotherapeutic agents include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide, and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin, and bizelesin synthetic analogues); podophyllotoxin; podophyllinic acid; teniposide; cryptophycins (particularly, cryptophycin 1 and cryptophycin 8); dolastatin; duocarmycin (including the synthetic analogues KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al. (1994) Angew. Chem Intl. Ed. Engl. 33: 183); dynemicin including dynemicin A; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, doxorubicin HCl liposome injection, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine, tegafur, capecitabine, an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially, T-2 toxin, verracurin A, roridin A, and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine and prednisolone, or FOLFOX, an abbreviation for a treatment regimen with oxaliplatin combined with 5-FU and leucovovin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Combination” as used herein refers to the provision of a first active modality in addition to one or more further active modalities (wherein one or more active modalities may be fused). Contemplated within the scope of the combinations described herein, are any regimen of combination modalities or partners (i.e., active compounds, components or agents), such as a combination of a PD-1 axis binding antagonist, a TGFβ inhibitor and a DNA-PK inhibitor, encompassed in single or multiple compounds and compositions. It is understood that any modalities within a single composition, formulation or unit dosage form (i.e., a fixed-dose combination) must have the identical dose regimen and route of delivery. It is not intended to imply that the modalities must be formulated for delivery together (e.g., in the same composition, formulation or unit dosage form). The combined modalities can be manufactured and/or formulated by the same or different manufacturers. The combination partners may thus be, e.g., entirely separate pharmaceutical dosage forms or pharmaceutical compositions that are also sold independently of each other. Preferably, the TGFβ inhibitor is fused to the PD-1 axis binding antagonist and therefore encompassed within a single composition and having an identical dose regimen and route of delivery.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, four or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject. Preferably, the combination therapies provided herein are used in conjunction with chemotherapy, radiotherapy or chemoradiotherapy.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Diabodies” refer to small antibody fragments prepared by constructing sFv fragments with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments, in which the V_{H} and V_{L} domains of the two antibodies are present on different polypeptide chains. Diabodies are described in greater detail in, for example, EP 404097; WO 1993/11161; Hollinger et al. (1993) PNAS USA 90: 6444.

“DNA-PK inhibitor” as used herein refers to a molecule that inhibits the activity of DNA-PK. Preferably, the DNA-PK inhibitor is (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof.

“Enhancing T-cell function” means to induce, cause or stimulate a T-cell to have a sustained or amplified biological function, or renew or reactivate exhausted or inactive T-cells. Examples of enhancing T-cell function include: increased secretion of y-interferon from CD8+ T-cells, increased proliferation, increased antigen responsiveness (e.g., viral, pathogen, or tumor clearance) relative to such levels before the intervention. In one embodiment, the level of. enhancement is as least 50%, alternatively 60%, 70%, 80%, 90%, 100%, 120%, 150%, 200%. The manner of measuring this enhancement is known to one of ordinary skill in the art.

“Fc” is a fragment comprising the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen-binding or variable region of the intact antibody or the Fc region of an antibody which retains or has modified FcR binding capability. Examples of functional antibody fragments include linear antibodies, single-chain antibody molecules, and multi-specific antibodies formed from antibody fragments.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227: 381; Marks et al. (1991) JMB 222: 581). Also available for the preparation of human monoclonal antibodies are methods described in Cole et al. (1985) Monoclonal Antibodies and Cancer Therapy, Alan R. Liss, page 77; Boerner et al. (1991), J. Immunol 147(1): 86; van Dijk and van de Winkel (2001) Curr. Opin. Pharmacol 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al. (2006) PNAS USA, 103: 3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321: 522; Riechmann et al. (1988), Nature 332: 323; Presta (1992) Curr. Op. Struct. Biol. 2: 593; Vaswani and Hamilton (1998), Ann. Allergy, Asthma & Immunol. 1: 105; Harris (1995) Biochem. Soc. Transactions 23: 1035; Hurle and Gross (1994) Curr. Op. Biotech. 5: 428; and U.S. Pat. Nos. 6,982,321 and 7,087,409.

“Immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1, and IgK1.

“Infusion” or “infusing” refers to the introduction of a drug-containing solution into the body through a vein for therapeutic purposes. Generally, this is achieved via an intravenous (IV) bag.

“Isolated” refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues. For example, an “isolated antibody” is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. The “isolated antibody” includes the antibody in-situ within recombinant cells since at least one component of the antibody's natural environment will not be present. Ordinarily, however, an isolated polypeptide or antibody will be prepared by at least one purification step.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256: 495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352: 624; Marks et al. (1992) JMB 222: 581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90: 2551; Jakobovits et al. (1993) Nature 362: 255; Bruggemann et al. (1993) Year in Immunol. 7: 33; U.S. Pat. Nos. 5,545,807; 5,545,806; 5,569,825; 5,625,126; 5,633,425; and U.S. Pat. No. 5,661,016; Marks et al. (1992) Bio/Technology 10: 779; Lonberg et al. (1994) Nature 368: 856; Morrison (1994) Nature 368: 812; Fishwild et al. (1996) Nature Biotechnol. 14: 845; Neuberger (1996), Nature Biotechnol. 14: 826; and Lonberg and Huszar (1995), Intern. Rev. Immunol. 13: 65-93). The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81: 6851).

“Nanobodies” refer to single-domain antibodies, which are fragments consisting of a single monomeric variable antibody domain. Like a whole antibody, they are able to bind selectively to a specific antigen. With a molecular weight of only 12-15 kDa, single-domain antibodies are much smaller than common antibodies (150-160 kDa). The first single-domain antibodies were engineered from heavy-chain antibodies found in camelids (see e.g., W. Wayt Gibbs, “Nanobodies”, Scientific American Magazine (August 2005)).

“Objective response” refers to a measurable response, including complete response (CR) or partial response (PR).

“Partial response” refers to a decrease in the size of one or more tumors or lesions, or in the extent of cancer in the body, in response to treatment.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate, a dog, cat, rabbit, pig, mouse, or rat, or animals used in screening, characterizing, and evaluating drugs and therapies.

“PD-1 axis binding antagonist” as used herein refers to a molecule that inhibits the interaction of PD-1 axis binding partners, such as PD-L1 and PD-1, to interfere with PD-1 signaling so as to remove T-cell dysfunction resulting from signaling on the PD-1 signaling axis, with a result being to restore or enhance T-cell function. As used herein, a PD-1 axis binding antagonist includes a PD-1 binding antagonist, a PD-L1 binding antagonist and a PD-L2 binding antagonist. In one embodiment, the PD-1 axis binding antagonist is an anti-PD-1 or anti-PD-L1 antibody, which is preferably fused to the TGFβ inhibitor. In one embodiment, the PD-L1 binding antagonist is the anti-PD-L1/TGFβ Trap molecule.

“PD-L1 expression” as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

“PD-L1 positive” cancer, including a “PD-L1 positive” cancerous disease, is one comprising cells, which have PD-L1 present at their cell surface. The term “PD-L1 positive” also refers to a cancer that produces sufficient levels of PD-L1 at the surface of cells thereof, such that an anti-PD-L1 antibody has a therapeutic effect, mediated by the binding of the said anti-PD-L1 antibody to PD-L1.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Serum” refers to the clear liquid that can be separated from clotted blood. Serum differs from plasma, the liquid portion of normal unclotted blood containing the red and white cells and platelets. Serum is the component that is neither a blood cell (serum does not contain white or red blood cells) nor a clotting factor. It is the blood plasma not including the fibrinogens that help in the formation of blood clots. It is the clot that makes the difference between serum and plasma.

“Single-chain Fv”, also abbreviated as “sFv” or “scFv”, are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

By “substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

“Suitable for therapy” or “suitable for treatment” shall mean that the patient is likely to exhibit one or more desirable clinical outcomes as compared to patients having the same cancer and receiving the same therapy but possessing a different characteristic that is under consideration for the purpose of the comparison. In one aspect, the characteristic under consideration is a genetic polymorphism or a somatic mutation (see e.g., Samsami et al. (2009) J Reproductive Med 54(1): 25). In another aspect, the characteristic under consideration is the expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same cancer and receiving the same therapy but not possessing the characteristic. As defined herein, the patient is considered suitable for the therapy. In another such aspect, a patient possessing a characteristic may exhibit one or more desirable clinical outcomes but simultaneously exhibit one or more less desirable clinical outcomes. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Systemic” treatment is a treatment, in which the drug substance travels through the bloodstream, reaching and affecting cells all over the body.

“TGFβ inhibitor” as used herein refers to a molecule that interferes with the interaction of the TGFβ ligand with its binding partners, such as the interaction between TGFβ and a TGFβ receptor (TGFβR), to inhibit the activity TGFβ. The TGFβ inhibitor may be TGFβ-binding antagonist or a TGFβR-binding antagonist. In one embodiment, the TGFβ inhibitor is fused to the PD-1 axis binding antagonist. In a further embodiment, an anti-PD-1 antibody or an anti-PD-L1 antibody is fused to the extracellular domain of a TGFβRII or a fragment of TGFβRII capable of binding TGFβ. In a particular embodiment the fusion protein comprises the heavy chains and light chains of SEQ ID NO: 3 and SEQ ID NO: 1, respectively, of WO 2015/118175. In another embodiment, the fusion protein is one of the fusion proteins disclosed in WO 2018/205985. In some embodiments, the fusion protein is one of the constructs listed in Table 2 of this publication, such as construct 9 or 15 thereof. In other embodiments, the antibody having the heavy chain sequence of SEQ ID NO: 11 and the light chain sequence of SEQ ID NO: 12 of WO 2018/205985 is fused via a linking sequence (G4S)xG, wherein x is 4-5, to the TGFβRII extracellular domain sequence of SEQ ID NO: 14 or SEQ ID NO: 15 of WO 2018/205985.

By “TGFβRII” or “TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO: 11)), or a polypeptide having the wild-type human TGFβ Receptor Type 2 Isoform B sequence (e.g., the amino acid sequence of NCBI RefSeq Accession No. NP_003233 (SEQ ID NO: 12)) or having a sequence substantially identical the amino acid sequence of SEQ ID NO: 11 or of SEQ ID NO: 12. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII lacks the signal sequence.

By a “fragment of TGFβRII capable of binding TGFβ” is meant any portion of NCBI RefSeq Accession No. NP_001020018 (SEQ ID NO: 11) or of NCBI RefSeq Accession No. NP_003233 (SEQ ID NO: 12), or a sequence substantially identical to SEQ ID NO: 11 or SEQ ID NO: 12 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 13.

“TGFβ expression” as used herein means any detectable level of expression of TGFβ protein or TGFβ mRNA within a cell or tissue. TGFβ protein expression may be detected with a diagnostic TGFβ antibody in an IHC assay of a tumor tissue section or by flow cytometry. Alternatively, TGFβ protein expression by tumor cells may be detected by PET imaging, using a binding agent (e.g., antibody fragment, affibody and the like) that specifically binds to TGFβ. Techniques for detecting and measuring TGFβ mRNA expression include RT-PCR and real-time quantitative RT-PCR.

“TGFβ positive” cancer, including a “TGFβ positive” cancerous disease, is one comprising cells, which secrete TGFβ. The term “TGFβ positive” also refers to a cancer that produces sufficient levels of TGFβ in the cells thereof, such that an TGFβ inhibitor has a therapeutic effect.

“Therapeutically effective amount” of a PD-1 axis binding antagonist, a TGFβ inhibitor or a DNA-PK inhibitor, in each case of the invention, refers to an amount effective, at dosages and for periods of time necessary, that, when administered to a patient with a cancer, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of a PD-1 axis binding antagonist, a TGFβ inhibitor or a DNA-PK inhibitor, to elicit a desired response in the individual. A therapeutically effective amount is also one in which any toxic or detrimental effects of a PD-1 axis binding antagonist, a TGFβ inhibitor or a DNA-PK inhibitor, are outweighed by the therapeutically beneficial effects.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al. (1991) Sequences of Immunological Interest, 5^{th} edition, National Institute of Health, Bethesda, Md.). The constant domains are not involved directly in the binding of antibody to an antigen, but exhibit various effector functions, such as participation of the antibody in antibody-dependent cellular toxicity.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Abbreviations

Some abbreviations used in the description include:

1L: First line
2L: Second line
ADCC: Antibody-dependent cell-mediated cytotoxicity
BID: Twice daily
CDR: Complementarity determining region
CR: Complete response
CRC: Colorectal cancer

### CRT: Chemoradiotherapy

### CT: Chemotherapy

DNA: Deoxyribonucleic acid
DNA-PK: DNA-dependent protein kinase
DNA-PKi: DNA-dependent protein kinase inhibitor
DSB: Double strand break
ED: Extensive disease

### Eto: Etoposide

### Ig: Immunoglobulin

### IHC: Immunohistochemistry

### IV: Intravenous

mCRC: Metastatic colorectal cancer
MSI-H: Microsatellite status instable high
MSI-L: Microsatellite status instable low
MSS: Microsatellite status stable
NK: Natural killers
NSCLC: Non-small-cell lung cancer
OS: Overall survival
PD: Progressive disease
PD-1: Programmed death 1
PD-L1: Programmed death ligand 1
PES: Polyester sulfone
PFS: Progression free survival
PR: Partial response
QD: Once daily
QID: Four times a day
Q2W: Every two weeks
Q3W: Every three weeks
RNA: Ribonucleic acid
RP2D: Recommended phase II dose
RR: Relative risk

### RT: Radiotherapy

SCCHN: Squamous cell carcinoma of the head and neck
SCLC: Small-cell lung cancer
SoC: Standard of care
SR: Sustained response
TID: Three times a day
TGFβ: Transforming growth factor β

### Topo: Topotecan

TR: Tumor response
TTP: Time to tumor progression
TTR: Time to tumor recurrence

### DESCRIPTIVE EMBODIMENTS

Therapeutic Combination and Method of Use Thereof

Some chemotherapies and radiotherapy can promote immunogenic tumor cell death and shape the tumor microenvironment to promote antitumor immunity. DNA-PK inhibition by means of DNA repair inhibitors can trigger and increase the immunogenic cell death induced by radiotherapy or chemotherapy and may therefore further increase T cell responses. The activation of the stimulator of interferon genes (STING) pathway and subsequent induction of type I interferons and PD-L1 expression is part of the response to double strand breaks in the DNA. Further, tumors with high somatic mutation burden are particularly responsive to checkpoint inhibitors, potentially due to increased neo-antigen formation. Particularly, there is a strong anti-PD1 response in mismatch repair-deficient CRC. DNA repair inhibitors may further increase the mutation rate of tumors and thus the repertoire of neo-antigens. Without being bound by any theory, the inventors assume that gathering double strand breaks (DSBs), e.g., by inhibiting DSB repair, particularly in combination with DNA-damaging interventions such as radiotherapy or chemotherapy, or in genetically instable tumors, sensitizes tumors to the treatment with a PD-1 axis binding antagonist, such as an anti-PD-L1 antibody comprising a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6, which is preferably fused to a TGFβ inhibitor. Inhibition of the interaction between PD-1 and PD-L1 enhances T-cell responses and mediates clinical antitumor activity. PD-1 is a key immune checkpoint receptor expressed by activated T cells, which mediates immunosuppression and functions primarily in peripheral tissues, where T cells may encounter the immunosuppressive PD-1 ligands PD-L1 (B7-H1) and PD-L2 (B7-DC), which are expressed by tumor cells, stromal cells, or both. Apart from upregulating PD-L1 expression, radiation therapy also causes increased levels of immunosuppressive cytokines like TGFβ, which attracts immune-suppressive cells into the tumor microenvironment.

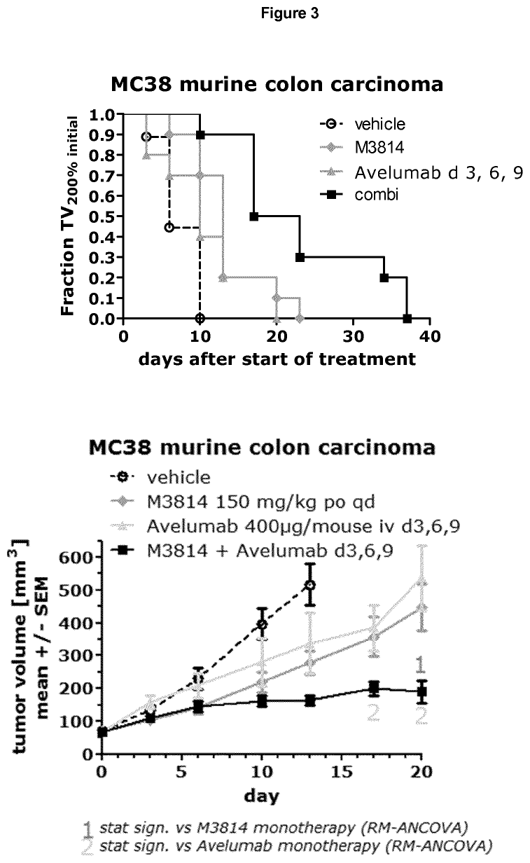

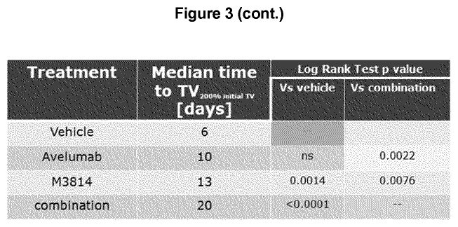

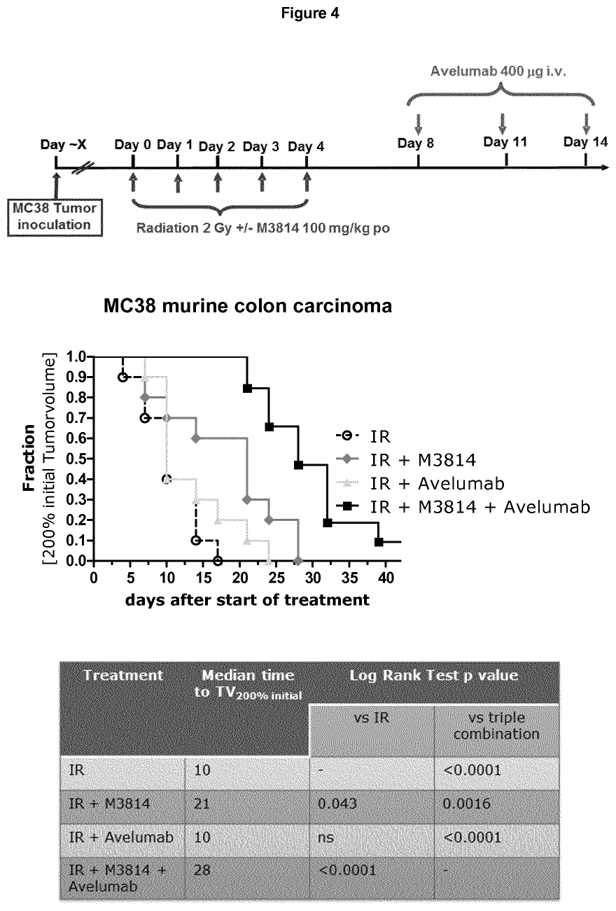

The present invention arose in part from the surprising discovery of a combination benefit for a DNA-PK inhibitor, a PD-1 axis binding antagonist and a TGFβ inhibitor, as well as for a DNA-PK inhibitor, a PD-1 axis binding antagonist and a TGFβ inhibitor in combination with radiotherapy, chemotherapy or chemoradiotherapy, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6. Adding a DNA-PK inhibitor to the said PD-1 axis binding antagonist was expected to be contraindicated, since DNA-PK is a major enzyme in VDJ recombination and as such potentially immunosuppressive to such an extent that deletion of DNA-PK leads to the SCID (severe combined immune deficiency) phenotype in mice. In contrast, the combination of the present invention delayed the tumor growth as compared to the single agent treatment. It was also not foreseeable that the further addition of a TGFβ inhibitor further inhibits tumor growth. Treatment schedule and doses were designed to reveal potential synergies. Pre-clinical data demonstrated a synergy of the DNA-PK inhibitor, particularly Compound 1, in combination with the PD-1 axis binding antagonist and the TGFβ inhibitor, particularly fused as the anti-PD-L1/TGFβ Trap molecule, optionally together with radiotherapy, versus the DNA-PK inhibitor or anti-PD-L1/TGFβ Trap (see e.g., FIG. 3 or 4).

Thus, in one aspect, the present invention provides a method for treating a cancer in a subject in need thereof, comprising administering to the subject a PD-1 axis binding antagonist, a TGFβ inhibitor and a DNA-PK inhibitor, preferably in combination with chemotherapy, radiotherapy or chemoradiotherapy. It shall be understood that a therapeutically effective amount of the PD-1 axis binding antagonist, TGFβ inhibitor and DNA-PK inhibitor is applied in the method of the invention, which is sufficient for treating one or more symptoms of a disease or disorder associated with PD-L1, TGFβ and DNA-PK, respectively.

Particularly, the present invention provides a method for treating a cancer in a subject in need thereof, comprising administering to the subject a PD-1 axis binding antagonist, a TGFβ inhibitor and a DNA-PK inhibitor, wherein the PD-1 axis binding antagonist is an anti-PD-L1 antibody and comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6, and is fused to the TGFβ inhibitor.

In one embodiment, the PD-1 axis binding antagonist is an anti-PD-L1 antibody, which is preferably a monoclonal antibody. In one embodiment, the anti-PD-L1 antibody exerts antibody-dependent cell-mediated cytotoxicity (ADCC). In one embodiment, the anti-PD-L1 antibody is a human or humanized antibody. In one embodiment, the anti-PD-L1 antibody is an isolated antibody. In a preferred embodiment, the anti-PD-L1 antibody is fused to the TGFβ inhibitor. In various embodiments, the anti-PD-L1 antibody is characterized by a combination of one or more of the foregoing features, as defined above.

In some embodiments, the PD-1 axis binding antagonist is an anti PD-L1 antibody selected from avelumab, durvalumab and atezolizumab. Avelumab is disclosed in International Patent Publication No. WO 2013/079174, the disclosure of which is hereby incorporated by reference in its entirety. Durvalumab is disclosed in International Patent Publication No. WO 2011/066389, the disclosure of which is hereby incorporated by reference in its entirety. Atezolizumab is disclosed in International Patent Publication No. WO 2010/077634, the disclosure of which is hereby incorporated by reference in its entirety.

In some embodiments, the PD-1 axis binding antagonist is an anti PD-1 antibody selected from nivolumab, pembrolizumab and cemiplimab. Nivolumab is disclosed in International Patent Publication No. WO 2006/121168, the disclosure of which is hereby incorporated by reference in its entirety. Pembrolizumab is disclosed in International Patent Publication No. WO 2008/156712, the disclosure of which is hereby incorporated by reference in its entirety.

Cemiplimab is disclosed in International Patent Publication No. WO 2015/112800, the disclosure of which is hereby incorporated by reference in its entirety.

In some embodiments, the PD-1 axis binding antagonist is the anti-PD-L1/TGFβ Trap molecule.

Further exemplary PD-1 axis binding antagonists for use in the treatment method, medicaments and uses of the present invention are mAb7 (aka RN888), mAb15, AMP224 and YW243.55.S70. mAb7 (aka RN888) and mAb15 are disclosed in International Patent Publication No. WO 2016/092419, the disclosure of which is hereby incorporated by reference in its entirety. AMP224 is disclosed in International Patent Publication No. WO 2010/027827 and WO 2011/066342, the disclosure of which is hereby incorporated by reference in its entirety. YW243.55.S70 is disclosed in International Patent Publication No. WO 2010/077634, the disclosure of which is hereby incorporated by reference in its entirety.

Further antibodies or agents that target PD-1 or PD-L1 are, e.g., CT-011 (Curetech), BMS-936559 (Bristol-Myers Squibb), MGA-271 (Macrogenics), dacarbazine and Lambrolizumab (MK-3475).

In various embodiments, the anti-PD-L1 antibody mediates antibody-dependent cell-mediated cytotoxicity (ADCC). In various embodiments, the anti-PD-L1 antibody is avelumab. Avelumab (formerly designated MSB0010718C) is a fully human monoclonal antibody of the immunoglobulin (Ig) G1 isotype (see e.g., WO 2013/079174). Avelumab selectively binds to PD-L1 and competitively blocks its interaction with PD-1. The mechanisms of action rely on the inhibition of PD-1/PD-L1 interaction and on natural killer (NK)-based ADCC (see e.g., Boyerinas et al. (2015) Cancer Immunol Res 3: 1148). Compared with anti-PD-1 antibodies that target T cells, avelumab targets tumor cells and therefore, it is expected to have fewer side effects, including a lower risk of autoimmune-related safety issues, as the blockade of PD-L1 leaves the PD-L2/PD-1 pathway intact to promote peripheral self-tolerance (see e.g., Latchman et al. (2001) Nat Immunol 2(3): 261).

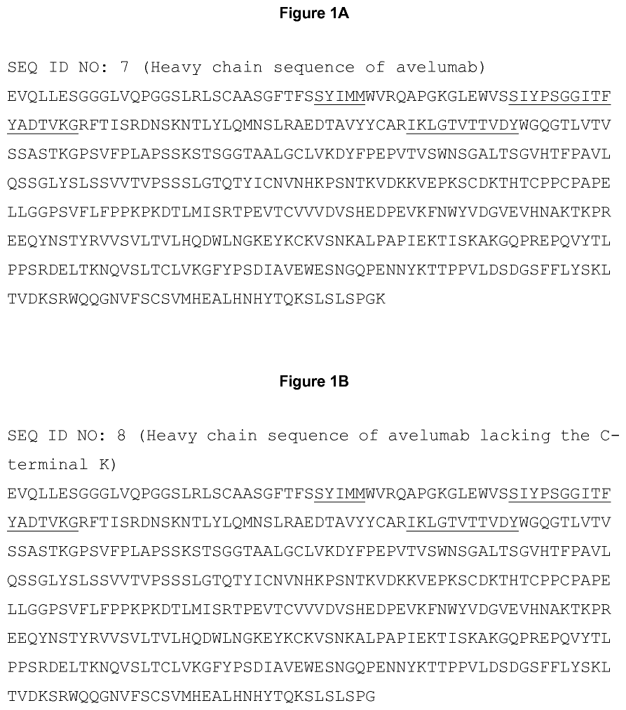

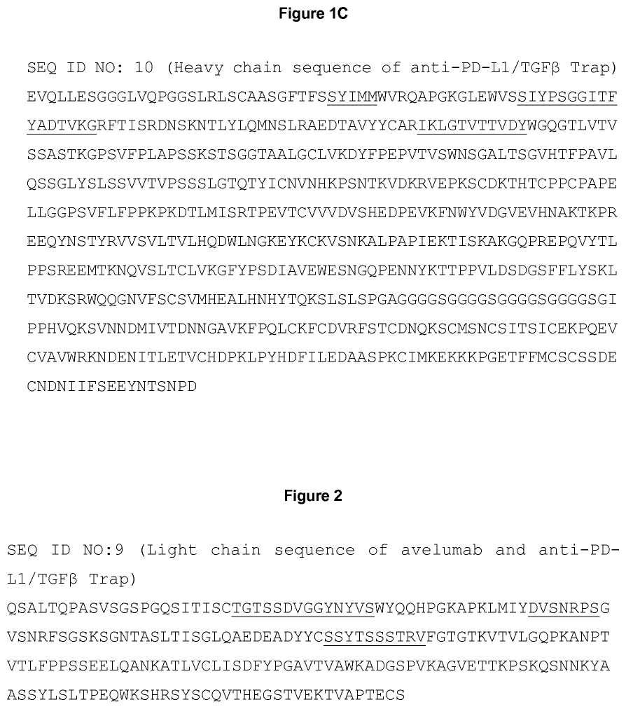

Avelumab, its sequence, and many of its properties have been described in WO 2013/079174, where it is designated A09-246-2 having the heavy and light chain sequences according to SEQ ID NOs: 32 and 33, as shown in FIG. 1 (SEQ ID NO: 7) and FIG. 2 (SEQ ID NO: 9), of this patent application. It is frequently observed, however, that in the course of antibody production the C-terminal lysine (K) of the heavy chain is cleaved off. This modification has no influence on the antibody-antigen binding. Therefore, in some embodiments the C-terminal lysine (K) of the heavy chain sequence of avelumab is absent. The heavy chain sequence of avelumab without the C-terminal lysine is shown in FIG. 1B (SEQ ID NO: 8), whereas FIG. 1A (SEQ ID NO: 7) shows the full length heavy chain sequence of avelumab. Further, as shown in WO 2013/079174, one of avelumab's properties is its ability to exert antibody-dependent cell-mediated cytotoxicity (ADCC), thereby directly acting on PD-L1 bearing tumor cells by inducing their lysis without showing any significant toxicity. In a preferred embodiment, the anti-PD-L1 antibody is avelumab, having the heavy and light chain sequences shown in FIG. 1A or 1B (SEQ ID NOs: 7 or 8), and FIG. 2 (SEQ ID NO: 9), or an antigen-binding fragment thereof.

In some embodiments, the TGFβ inhibitor is selected from the group consisting of a TGFβ receptor, a TGFβ ligand- or receptor-blocking antibody, a small molecule inhibiting the interaction between TGFβ binding partners and an inactive mutant TGFβ ligand that binds to the TGFβ receptor and competes for binding with endogenous TGFβ. Preferably, the TGFβ inhibitor is a TGFβ receptor or a fragment thereof capable of binding TGFβ.

Exemplary TGFβ ligand-blocking antibodies include lerdelimumab, metelimumab, fresolimumab, XPA681, XPA089 and LY2382770. Exemplary TGFβ receptor-blocking antibodies include 1D11, 2G7, GC1008 and LY3022859.

In some aspects, the DNA-PK inhibitor is (S)-[2-chloro-4-fluoro-5-(7-morpholin-4-yl-quinazolin-4-yl)-phenyl]-(6-methoxypyridazin-3-yl)-methanol, having the structure of Compound 1:

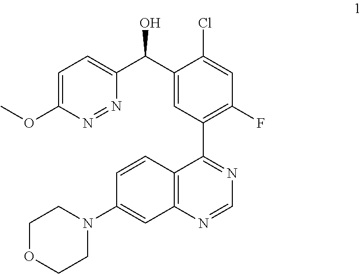 [SMILES: COc1ccc([C@@H](O)c2cc(-c3ncnc4cc(N5CCOCC5)ccc34)c(F)cc2Cl)nn1]

or a pharmaceutically acceptable salt thereof.

Compound 1 is described in detail in United States patent application US 2016/0083401, published on Mar. 24, 2016 (referred to herein as “the '401 publication”), the entirety of which is hereby incorporated herein by reference. Compound 1 is designated as compound 136 in Table 4 of the '401 publication. Compound 1 is active in a variety of assays and therapeutic models demonstrating inhibition of DNA-PK (see, e.g., Table 4 of the '401 publication). Accordingly, Compound 1, or a pharmaceutically acceptable salt thereof, is useful for treating one or more disorders associated with activity of DNA-PK, as described in detail herein.

Compound 1 is a potent and selective ATP-competitive inhibitor of DNA-PK, as demonstrated by crystallographic and enzyme kinetics studies. DNA-PK, together with five additional protein factors (Ku70, Ku80, XRCC4, Ligase IV and Artemis) plays a critical role in the repair of DSB via NHEJ. Kinase activity of DNA-PK is essential for proper and timely DNA repair and the long-term survival of cancer cells. Without wishing to be bound by any particular theory, it is believed that the primary effects of Compound 1 are suppression of DNA-PK activity and DNA double strand break (DSB) repair, leading to altered repair of DNA and potentiation of antitumor activity of DNA-damaging agents.

It is understood that although the methods described herein may refer to formulations, doses and dosing regimens/schedules of Compound 1, such formulations, doses and/or dosing regimens/schedules are equally applicable to any pharmaceutically acceptable salt of Compound 1. Accordingly, in some embodiments, a dose or dosing regimen for a pharmaceutically acceptable salt of Compound 1, or a pharmaceutically acceptable salt thereof, is selected from any of the doses or dosing regimens for Compound 1 as described herein.

A pharmaceutically acceptable salt may involve the inclusion of another molecule, such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In one embodiment, the therapeutic combination of the invention is used in the treatment of a human subject. In one embodiment, the anti-PD-L1 antibody targets PD-L1 which is human PD-L1. The main expected benefit in the treatment with the therapeutic combination is a gain in risk/benefit ratio with said antibody, particularly avelumab or anti-PD-L1/TGFβ Trap, for these human patients.

In one embodiment, the cancer is identified as a PD-L1 positive cancerous disease. Pharmacodynamic analyses show that tumor expression of PD-L1 might be predictive of treatment efficacy. According to the invention, the cancer is preferably considered to be PD-L1 positive if between at least 0.1% and at least 10% of the cells of the cancer have PD-L1 present at their cell surface, more preferably between at least 0.5% and 5%, most preferably at least 1%. In one embodiment, the PD-L1 expression is determined by immunohistochemistry (IHC).

In certain embodiments, the invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In another embodiment, the cancer is selected from cancer of the lung, head and neck, colon, neuroendocrine system, mesenchyme, breast, ovarian, pancreatic, gastric, esophageal, glioblastoma and histological subtypes thereof (e.g., adeno, squamous, large cell). In a preferred embodiment, the cancer is selected from small-cell lung cancer (SCLC), non-small-cell lung cancer (NSCLC), squamous cell carcinoma of the head and neck (SCCHN), colorectal cancer (CRC), primary neuroendocrine tumors and sarcoma.

In various embodiments, the method of the invention is employed as a first, second, third or later line of treatment. A line of treatment refers to a place in the order of treatment with different medications or other therapies received by a patient. First-line therapy regimens are treatments given first, whereas second- or third-line therapy is given after the first-line therapy or after the second-line therapy, respectively. Therefore, first-line therapy is the first treatment for a disease or condition. In patients with cancer, first-line therapy, sometimes referred to as primary therapy or primary treatment, can be surgery, chemotherapy, radiation therapy, or a combination of these therapies. Typically, a patient is given a subsequent chemotherapy regimen (second- or third-line therapy), either because the patient did not show a positive clinical outcome or only showed a sub-clinical response to a first- or second-line therapy or showed a positive clinical response but later experienced a relapse, sometimes with disease now resistant to the earlier therapy that elicited the earlier positive response.

If the safety and the clinical benefit offered by the therapeutic combination of the invention are confirmed, this combination of a PD-1 axis binding antagonist, a TGFβ inhibitor and a DNA-PK inhibitor warrants a first-line setting in cancer patients. Particularly, the combination may become a new standard treatment for patients suffering from a cancer that is selected from the group of SCLC extensive disease (ED), NSCLC and SCCHN.

It is preferred that the therapeutic combination of the invention is applied in a later line of treatment, particularly a second-line or higher treatment of the cancer. There is no limitation to the prior number of therapies provided that the subject underwent at least one round of prior cancer therapy. The round of prior cancer therapy refers to a defined schedule/phase for treating a subject with, e.g., one or more chemotherapeutic agents, radiotherapy or chemoradiotherapy, and the subject failed with such previous treatment, which was either completed or terminated ahead of schedule. One reason could be that the cancer was resistant or became resistant to prior therapy. The current standard of care (SoC) for treating cancer patients often involves the administration of toxic and old chemotherapy regimens. The SoC is associated with high risks of strong adverse events that are likely to interfere with the quality of life (such as secondary cancers). The toxicity profile of an anti-PD-L1 antibody/DNA-PK inhibitor combination, preferably avelumab and (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof, seems to be much better than the SoC chemotherapy. In one embodiment, an anti-PD-L1 antibody/DNA-PK inhibitor combination, preferably avelumab and (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof, may be as effective and better tolerated than SoC chemotherapy in patients with cancer resistant to mono- and/or poly-chemotherapy, radiotherapy or chemoradiotherapy.

As the modes of action of the DNA-PK inhibitor, the PD-1 axis binding antagonist and the TGFβ inhibitor are different, it is thought that the likelihood that administration of the therapeutic treatment of the invention may lead to enhanced immune-related adverse events (irAE) is small although all three agents are targeting the immune system.

In a preferred embodiment, the DNA-PK inhibitor, the PD-1 axis binding antagonist and the TGFβ inhibitor are administered in a second-line or higher treatment, more preferably a second-line treatment, of the cancer selected from the group of pre-treated relapsing metastatic NSCLC, unresectable locally advanced NSCLC, pre-treated SCLC ED, SCLC unsuitable for systemic treatment, pre-treated relapsing (recurrent) or metastatic SCCHN, recurrent SCCHN eligible for re-irradiation, and pre-treated microsatellite status instable low (MSI-L) or microsatellite status stable (MSS) metastatic colorectal cancer (mCRC). SCLC and SCCHN are particularly systemically pre-treated. MSI-L/MSS mCRC occurs in 85% of all mCRC. Once, the safety/tolerability and efficacy profile of the combination of the DNA-PK inhibitor, the PD-1 axis binding antagonist and the TGFβ inhibitor is established in patients, using, e.g., the standard dose of the anti-PD-L1/TGFβ Trap molecule and the recommended phase II dose (RP2D) of the DNA-PK inhibitor, in each case as described herein, additional expansion cohorts including chemotherapy (e.g., etoposide or topotecan), radiotherapy or chemoradiotherapy to introduce double-strand breaks are targeted.

In some embodiments that employ an anti-PD-L1 antibody in the combination therapy, the dosing regimen will comprise administering the anti-PD-L1 antibody at a dose of about 1, 2, 3, 4, 5, 6, 7, 8, 9, 10, 11, 12, 13, 14, 15, 16, 17, 18, 19 or 20 mg/kg at intervals of about 14 days (±2 days) or about 21 days (±2 days) or about 30 days (±2 days) throughout the course of treatment. In other embodiments that employ an anti-PD-L1 antibody in the combination therapy, the dosing regimen will comprise administering the anti-PD-L1 antibody at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose. In certain embodiments, a subject will be administered an intravenous (IV) infusion of a medicament comprising any of the anti-PD-L1 antibody described herein. In some embodiments, the anti-PD-L1 antibody in the combination therapy is avelumab, which is administered intravenously at a dose selected from the group consisting of: about 1 mg/kg Q2W (Q2W=one dose every two weeks), about 2 mg/kg Q2W, about 3 mg/kg Q2W, about 5 mg/kg Q2W, about 10 mg/kg Q2W, about 1 mg/kg Q3W (Q3W=one dose every three weeks), about 2 mg/kg Q3W, about 3 mg/kg Q3W, about 5 mg/kg Q3W, and about 10 mg Q3W. In some embodiments of the invention, the anti-PD-L1 antibody in the combination therapy is avelumab, which is administered in a liquid medicament at a dose selected from the group consisting of about 1 mg/kg Q2W, about 2 mg/kg Q2W, about 3 mg/kg Q2W, about 5 mg/kg Q2W, about 10 mg/kg Q2W, about 1 mg/kg Q3W, about 2 mg/kg Q3W, about 3 mg/kg Q3W, about 5 mg/kg Q3W, and about 10 mg/kg Q3W. In some embodiments, a treatment cycle begins with the first day of combination treatment and last for 2 weeks. In such embodiments, the combination therapy is preferably administered for at least 12 weeks (6 cycles of treatment), more preferably at least 24 weeks, and even more preferably at least 2 weeks after the patient achieves a CR.

In some embodiments that employ an anti-PD-L1 antibody in the combination therapy, the dosing regimen will comprise administering the anti-PD-L1 antibody at a dose of about 400-800 mg flat dose Q2W. Preferably, the flat dosing regimen is 400 mg, 450 mg, 500 mg, 550 mg, 600 mg, 650 mg, 700 mg 750 mg or 800 mg flat dose Q2W. More preferably, the flat dosing regimen is 800 mg flat dose Q2W. In some more preferred embodiments that employ an anti-PD-L1 antibody in the combination therapy, the dosing regimen will be a fixed dose of 800 mg given intravenously at intervals of about 14 days (±2 days).

In another embodiment, the anti-PD-L1 antibody, preferably avelumab, will be given IV every two weeks (Q2W). In certain embodiments, the anti-PD-L1 antibody is administered intravenously for 50-80 minutes at a dose of about 10 mg/kg body weight every two weeks (Q2W). In a more preferred embodiment, the avelumab dose will be 10 mg/kg body weight administered as 1-hour intravenous infusions every two weeks (Q2W). In certain embodiments, the anti-PD-L1 antibody is administered intravenously for 50-80 minutes at a fixed dose of about 800 mg every two weeks (Q2W). In a more preferred embodiment, the avelumab dose will be 800 mg administered as 1-hour intravenous infusions every 2 weeks (Q2W). Given the variability of infusion pumps from site to site, a time window of minus 10 minutes and plus 20 minutes is permitted.

Pharmacokinetic studies demonstrated that the 10 mg/kg dose of avelumab achieves excellent receptor occupancy with a predictable pharmacokinetics profile (see e.g., Heery et al. (2015) Proc 2015 ASCO Annual Meeting, abstract 3055). This dose is well tolerated, and signs of antitumor activity, including durable responses, have been observed. Avelumab may be administered up to 3 days before or after the scheduled day of administration of each cycle due to administrative reasons. Pharmacokinetic simulations also suggested that exposures to avelumab across the available range of body weights are less variable with 800 mg Q2W compared with 10 mg/kg Q2W. Exposures were similar near the population median weight. Low-weight subjects tended towards marginally lower exposures relative to the rest of the population when weight based dosing was used, and marginally higher exposures when flat dosing was applied. The implications of these exposure differences are not expected to be clinically meaningful at any weight across the whole population. Furthermore, the 800 mg Q2W dosing regimen is expected to result in C_{trough}>1 mg/mL required to maintain avelumab serum concentrations at >95% TO throughout the entire Q2W dosing interval in all weight categories. In a preferred embodiment, a fixed dosing regimen of 800 mg administered as a 1 hour IV infusion Q2W will be utilized for avelumab in clinical trials.

In certain embodiments that employ an anti-PD-L1/TGFβ Trap in the combination therapy, the dosing regimen comprises administering the anti-PD-L1/TGFβ Trap at a dose of about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200, about 1300, about 1400, about 1500, about 1600, about 1700, about 1800, about 1900, about 2000, about 2100, about 2200, about 2300, about 2400, about 2500 mg, about 2600 mg, about 2700 mg, about 2800 mg, about 2900 mg, or about 3000 mg). In certain embodiments, about 1200 mg of anti-PD-L1/TGFβ Trap molecule is administered to a subject once every two weeks. In certain embodiments, about 1800 mg of anti-PD-L1/TGFβ Trap molecule is administered to a subject once every three weeks. In certain embodiments, about 2400 mg of anti-PD-L1/TGFβ Trap molecule is administered to a subject once every three weeks. In certain embodiments, about 1200 mg of a protein product with a first polypeptide that includes the amino acid sequence of SEQ ID NO: 10 and the second polypeptide that includes the amino acid sequence of SEQ ID NO: 9 is administered to a subject once every two weeks. In certain embodiments, about 1800 mg of a protein product with a first polypeptide that includes the amino acid sequence of SEQ ID NO: 10 and the second polypeptide that includes the amino acid sequence of SEQ ID NO: 9 is administered to a subject once every three weeks. In certain embodiments, about 2400 mg of a protein product with a first polypeptide that includes the amino acid sequence of SEQ ID NO: 10 and the second polypeptide that includes the amino acid sequence of SEQ ID NO: 9 is administered to a subject once every three weeks.

In some embodiments, provided methods comprise administering a pharmaceutically acceptable composition comprising the DNA-PK inhibitor, preferably Compound 1, or a pharmaceutically acceptable salt thereof, one, two, three or four times a day. In some embodiments, a pharmaceutically acceptable composition comprising the DNA-PK inhibitor, preferably Compound 1, or a pharmaceutically acceptable salt thereof, is administered once daily (“QD”), particularly continuously. In some embodiments, a pharmaceutically acceptable composition comprising the DNA-PK inhibitor, preferably Compound 1, or a pharmaceutically acceptable salt thereof, is administered twice daily, particularly continuously. In some embodiments, twice daily administration refers to a compound or composition that is administered “BID”, or two equivalent doses administered at two different times in one day. In some embodiments, a pharmaceutically acceptable composition comprising the DNA-PK inhibitor, preferably Compound 1, or a pharmaceutically acceptable salt thereof, is administered three times a day. In some embodiments, a pharmaceutically acceptable composition comprising Compound 1, or a pharmaceutically acceptable salt thereof, is administered “TID”, or three equivalent doses administered at three different times in one day. In some embodiments, a pharmaceutically acceptable composition comprising the DNA-PK inhibitor, preferably Compound 1, or a pharmaceutically acceptable salt thereof, is administered four times a day. In some embodiments, a pharmaceutically acceptable composition comprising Compound 1, or a pharmaceutically acceptable salt thereof, is administered “QID”, or four equivalent doses administered at four different times in one day. In some embodiments, the DNA-PK inhibitor, preferably Compound 1, or a pharmaceutically acceptable salt thereof, is administered to a patient under fasted conditions and the total daily dose is any of those contemplated above and herein. In some embodiments, the DNA-PK inhibitor, preferably Compound 1, or a pharmaceutically acceptable salt thereof, is administered to a patient under fed conditions and the total daily dose is any of those contemplated above and herein. In some embodiments, the DNA-PK inhibitor, preferably Compound 1, or a pharmaceutically acceptable salt thereof, is administered orally. In some embodiments, the DNA-PK inhibitor, preferably Compound 1, or a pharmaceutically acceptable salt thereof, will be given orally either once or twice daily continuously. In preferred embodiments, the DNA-PK inhibitor, preferably Compound 1, or a pharmaceutically acceptable salt thereof, is administered once daily (QD) or twice daily (BID), at a dose of about 1 to about 800 mg. In preferred embodiments, the DNA-PK inhibitor, preferably Compound 1, or a pharmaceutically acceptable salt thereof, is administered twice daily (BID), at a dose of about 400 mg.

Concurrent treatment considered necessary for the patient's well-being may be given at discretion of the treating physician. In some embodiments, the PD-1 axis binding antagonist, TGFβ inhibitor and DNA-PK inhibitor are administered in combination with chemotherapy (CT), radiotherapy (RT), or chemotherapy and radiotherapy (CRT). As described herein, in some embodiments, the present invention provides methods of treating, stabilizing or decreasing the severity or progression of one or more diseases or disorders associated with PD-L1, TGFβ and DNA-PK comprising administering to a patient in need thereof a PD-1 axis binding antagonist, a TGFβ inhibitor and an inhibitor of DNA-PK in combination with an additional chemotherapeutic agent. In certain embodiments, the chemotherapeutic agent is selected from the group of etoposide, doxorubicin, topotecan, irinotecan, fluorouracil, a platin, an anthracycline, and a combination thereof.

In certain embodiments, the additional chemotherapeutic agent is etoposide. Etoposide forms a ternary complex with DNA and the topoisomerase II enzyme which aids in DNA unwinding during replication. This prevents re-ligation of the DNA strands and causes DNA strands to break. Cancer cells rely on this enzyme more than healthy cells because they divide more rapidly. Therefore, etoposide treatment causes errors in DNA synthesis and promotes apoptosis of the cancer cells. Without wishing to be bound by any particular theory, it is believed that a DNA-PK inhibitor blocks one of the main pathways for repair of DSBs in DNA thus delaying the repair process and leading to an enhancement of the antitumor activity of etoposide. In-vitro data demonstrated a synergy of Compound 1 in combination with etoposide versus etoposide alone. Thus, in some embodiments, a provided combination of Compound 1, or a pharmaceutically acceptable salt thereof, with etoposide is synergistic.

In certain embodiments, the additional chemotherapeutic agent is topotecan, etoposide and/or anthracycline treatment, either as single cytostatic agent or as part of a doublet or triplet regiment. With such a chemotherapy, the DNA-PK inhibitor can be preferably given once or twice daily with the PD-1 axis binding antagonist and TGFβ inhibitor, preferably fused as anti-PD-L1/TGFβ Trap, which is given given once every two weeks or once every three weeks. In cases, in which anthracyclines are used, the treatment with anthracycline is stopped once a maximal life-long accumulative dose has been reached (due to the cardiotoxicity).

In certain embodiments, the additional chemotherapeutic agent is a platin. Platins are platinum-based chemotherapeutic agents. As used herein, the term “platin” is used interchangeably with the term “platinating agent.” Platinating agents are well known in the art. In some embodiments, the platin (or platinating agent) is selected from cisplatin, carboplatin, oxaliplatin, nedaplatin, and satraplatin. In some embodiments, the additional chemotherapeutic is a combination of both of etoposide and a platin. In certain embodiments, the platin is cisplatin. In certain embodiments, the provided method further comprises administration of radiation therapy to the patient. In some embodiments, the additional chemotherapeutic is a combination of both of etoposide and cisplatin.

In certain embodiments, the additional therapeutic agent is selected from daunomycin, doxorubicin, epirubicin, idarubicin, valrubicin, mitoxantrone, paclitaxel, docetaxel and cyclophosphamide.

In other embodiments, the additional therapeutic agent is selected from a CTLA4 agent (e.g., ipilimumab (BMS)); GITR agent (e.g., MK-4166 (MSD)); vaccines (e.g., sipuleucel-t (Dendron); or a SoC agent (e.g., radiation, docetaxel, temozolomide (MSD), gemcitibine or paclitaxel). In other embodiments, the additional therapeutic agent is an immune enhancer such as a vaccine, immune-stimulating antibody, immunoglobulin, agent or adjuvant including, but not limited to, sipuleucel-t, BMS-663513 (BMS), CP-870893 (Pflzer/VLST), anti-OX40 (AgonOX), or CDX-1127 (CellDex).

Other cancer therapies or anticancer agents that may be used in combination with the inventive agents of the present invention include surgery, radiotherapy (e.g., gamma-radiation, neutron beam radiotherapy, electron beam radiotherapy, proton therapy, brachytherapy, low-dose radiotherapy, and systemic radioactive isotopes), immune response modifiers such as chemokine receptor antagonists, chemokines and cytokines (e.g., interferons, interleukins, tumor necrosis factor (TNF), and GM-CSF)), hyperthermia and cryotherapy, agents to attenuate any adverse effects (e.g. antimetics, steroids, anti-inflammatory agents), and other approved chemotherapeutic drugs.

In certain embodiments, the additional therapeutic agent is selected from an antibiotic, a vasopressor, a steroid, an inotrope, an anti-thrombotic agent, a sedative, opioids or an anesthetic.

In certain embodiments, the additional therapeutic agent is selected from cephalosporins, macrolides, penams, beta-lactamase inhibitors, aminoglycoside antibiotics, fluoroquinolone antibiotics, glycopeptide antibiotics, penems, monobactams, carbapenmems, nitroimidazole antibiotics, lincosamide antibiotics, vasopressors, positive inotropic agents, steroids, benzodiazepines, phenol, alpha2-adrenergic receptor agonists, GABA-A receptor modulators, anti-thrombotic agents, anesthetics or opiods.

The DNA-PK inhibitor, preferably Compound 1, or a pharmaceutically acceptable salt thereof, and compositions thereof in combination with the PD-1 axis binding antagonist, TGFβ inhibitor and additional chemotherapeutic according to methods of the present invention, are administered using any amount and any route of administration effective for treating or decreasing the severity of a disorder provided above. The exact amount required will vary from subject to subject, depending on the species, age, and general condition of the subject, the severity of the infection, the particular agent, its mode of administration, and the like.

In some embodiments, the present invention provides a method of treating a cancer selected from lung, head and neck, colon, neuroendocrine system, mesenchyme, breast, ovarian, pancreatic, and histological subtypes thereof (e.g., adeno, squamous, large cell) in a patient in need thereof comprising administering to said patient the DNA-PK inhibitor, preferably Compound 1, or a pharmaceutically acceptable salt thereof, in an amount of about 1 to about 800 mg, preferably in an amount of about 10 to about 800 mg, more preferably in an amount of about 100 to about 400 mg, in each case in combination with the PD-1 axis binding antagonist, TGFβ inhibitor and at least one additional therapeutic agent selected from a platin and etoposide, in amounts according to the local clinical standard of care guidelines.

In some embodiments, provided methods comprise administering a pharmaceutically acceptable composition comprising a chemotherapeutic agent one, two, three or four times a day. In some embodiments, a pharmaceutically acceptable composition comprising a chemotherapeutic agent is administered once daily (“QD”). In some embodiments, a pharmaceutically acceptable composition comprising a chemotherapeutic agent is administered twice daily. In some embodiments, twice daily administration refers to a compound or composition that is administered “BID”, or two equivalent doses administered at two different times in one day. In some embodiments, a pharmaceutically acceptable composition comprising a chemotherapeutic agent is administered three times a day. In some embodiments, a pharmaceutically acceptable composition comprising a chemotherapeutic agent is administered “TID”, or three equivalent doses administered at three different times in one day. In some embodiments, a pharmaceutically acceptable composition comprising a chemotherapeutic agent is administered four times a day. In some embodiments, a pharmaceutically acceptable composition comprising a chemotherapeutic agent is administered

“QID”, or four equivalent doses administered at four different times in one day. In some embodiments, a pharmaceutically acceptable composition comprising a chemotherapeutic agent is administered for a various number of days (for example 14, 21, 28) with a various number of days between treatment (0, 14, 21, 28). In some embodiments, a chemotherapeutic agent is administered to a patient under fasted conditions and the total daily dose is any of those contemplated above and herein. In some embodiments, a chemotherapeutic agent is administered to a patient under fed conditions and the total daily dose is any of those contemplated above and herein. In some embodiments, a chemotherapeutic agent is administered orally for reasons of convenience. In some embodiments, when administered orally, a chemotherapeutic agent is administered with a meal and water. In another embodiment, the chemotherapeutic agent is dispersed in water or juice (e.g., apple juice or orange juice) and administered orally as a suspension. In some embodiments, when administered orally, a chemotherapeutic agent is administered in a fasted state. A chemotherapeutic agent can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In certain embodiments, the PD-1 axis binding antagonist, TGFβ inhibitor and DNA-PK inhibitor, preferably Compound 1, or a pharmaceutically acceptable salt thereof, are administered in combination with radiotherapy. In certain embodiments, provided methods comprise administration of the PD-1 axis binding antagonist, TGFβ inhibitor and DNA-PK inhibitor, preferably Compound 1, or a pharmaceutically acceptable salt thereof, in combination with one or both of etoposide and cisplatin, wherein said method further comprises administering radiotherapy to the patient. In certain embodiments, the radiotherapy comprises about 35-70 Gy/20-35 fractions. In some embodiments, the radiotherapy is given either with standard fractionation (1.8 to 2 Gy per day for 5 days a week) up to a total dose of 50-70 Gy. Other fractionation schedules could also be envisioned, for example, a lower dose per fraction but given twice daily with the DNA-PK inhibitor given also twice daily. Higher daily doses over a shorter period of time can also be given. In one embodiment, stereotactic radiotherapy as well as the gamma knife are used. In the palliative setting, other fractionation schedules are also widely used for example 25 Gy in 5 fractions or 30 Gy in 10 fractions. In all cases, anti-PD-L1/TGFβ Trap is preferably given once every two weeks or once every three weeks. For radiotherapy, the duration of treatment will be the time frame when radiotherapy is given. These interventions apply to treatment given with electrons, photons and protons, alfa-emitters or other ions, treatment with radio-nucleotides, for example, treatment with ¹³¹I given to patients with thyroid cancer, as well in patients treated with boron capture neutron therapy.

In some embodiments, the PD-1 axis binding antagonist, TGFβ inhibitor and DNA-PK inhibitor are administered simultaneously, separately or sequentially and in any order. The PD-1 axis binding antagonist, TGFβ inhibitor and DNA-PK inhibitor are administered to the patient in any order (i.e., simultaneously or sequentially) in separate compositions, formulations or unit dosage forms, or together in a single composition, formulation or unit dosage form. In one embodiment, a method of treating a proliferative disease may comprise administration of a combination of a DNA-PK inhibitor, a TGFβ inhibitor and a PD-1 axis binding antagonist, wherein the individual combination partners are administered simultaneously or sequentially in any order, in jointly therapeutically effective amounts, (for example in synergistically effective amounts), e.g. in daily or intermittently dosages corresponding to the amounts described herein. The individual combination partners of a combination therapy of the invention may be administered separately at different times during the course of therapy or concurrently in divided or single combination forms. Typically, in such combination therapies, the first active component which is at least one DNA-PK inhibitor, and the PD-1 axis binding antagonist and TGFβ inhibitor are formulated into separate pharmaceutical compositions or medicaments. When separately formulated, the at least three active components can be administered simultaneously or sequentially, optionally via different routes. Optionally, the treatment regimens for each of the active components in the combination have different but overlapping delivery regimens, e.g., daily, twice daily, vs. a single administration, or weekly. The second and third active component (PD-1 axis binding antagonist and TGFβ inhibitor) may independently from one another be delivered prior to, substantially simultaneously with, or after, the at least one DNA-PK inhibitor. In certain embodiments, the PD-1 axis binding antagonist, TGFβ inhibitor and DNA-PK inhibitor are administered simultaneously in the same composition comprising the PD-1 axis binding antagonist, TGFβ inhibitor and DNA-PK inhibitor. In certain embodiments, the PD-1 axis binding antagonist, TGFβ inhibitor and DNA-PK inhibitor are administered simultaneously in separate compositions, i.e., wherein the PD-1 axis binding antagonist, TGFβ inhibitor and DNA-PK inhibitor are administered simultaneously each in a separate unit dosage form. It will be appreciated that the PD-1 axis binding antagonist, TGFβ inhibitor and DNA-PK inhibitor are administered on the same day or on different days and in any order as according to an appropriate dosing protocol. The instant invention is therefore to be understood as embracing all such regimens of simultaneous or alternating treatment and the term “administering” is to be interpreted accordingly.

In some embodiments, the anti-PD-L1/TGFβ Trap and DNA-PK inhibitor are administered simultaneously, separately or sequentially and in any order. The anti-PD-L1/TGFβ Trap and DNA-PK inhibitor are administered to the patient in any order (i.e., simultaneously or sequentially) in separate compositions, formulations or unit dosage forms, or together in a single composition, formulation or unit dosage form. In one embodiment, a method of treating a proliferative disease may comprise administration of a combination of a DNA-PK inhibitor and an anti-PD-L1/TGFβ Trap, wherein the individual combination partners are administered simultaneously or sequentially in any order, in jointly therapeutically effective amounts, (for example in synergistically effective amounts), e.g. in daily or intermittently dosages corresponding to the amounts described herein. The individual combination partners of a combination therapy of the invention may be administered separately at different times during the course of therapy or concurrently in divided or single combination forms. Typically, in such combination therapies, the first active component which is at least one DNA-PK inhibitor, and the anti-PD-L1/TGFβ Trap are formulated into separate pharmaceutical compositions or medicaments. When separately formulated, the at least two active components can be administered simultaneously or sequentially, optionally via different routes. Optionally, the treatment regimens for each of the active components in the combination have different but overlapping delivery regimens, e.g., daily, twice daily, vs. a single administration, or weekly. The second active component (anti-PD-L1/TGFβ Trap) may be delivered prior to, substantially simultaneously with, or after, the at least one DNA-PK inhibitor. In certain embodiments, the anti-PD-L1/TGFβ Trap is administered simultaneously in the same composition comprising the anti-PD-L1/TGFβ Trap and DNA-PK inhibitor. In certain embodiments, the anti-PD-L1/TGFβ Trap and DNA-PK inhibitor are administered simultaneously in separate compositions, i.e., wherein the anti-PD-L1/TGFβ Trap and DNA-PK inhibitor are administered simultaneously each in a separate unit dosage form. It will be appreciated that the anti-PD-L1/TGFβ Trap and DNA-PK inhibitor are administered on the same day or on different days and in any order as according to an appropriate dosing protocol. The instant invention is therefore to be understood as embracing all such regimens of simultaneous or alternating treatment and the term “administering” is to be interpreted accordingly.

In some embodiments, the combination regimen comprises the steps of: (a) under the direction or control of a physician, the subject receiving the PD-1 axis binding antagonist and TGFβ inhibitor prior to first receipt of the DNA-PK inhibitor; and (b) under the direction or control of a physician, the subject receiving the DNA-PK inhibitor. In some embodiments, the combination regimen comprises the steps of: (a) under the direction or control of a physician, the subject receiving the DNA-PK inhibitor prior to first receipt of the PD-1 axis binding antagonist and TGFβ inhibitor; and (b) under the direction or control of a physician, the subject receiving the PD-1 axis binding antagonist and TGFβ inhibitor. In some embodiments, the combination regimen comprises the steps of: (a) prescribing the subject to self-administer, and verifying that the subject has self-administered, the PD-1 axis binding antagonist and TGFβ inhibitor prior to first administration of the DNA-PK inhibitor; and (b) administering the DNA-PK inhibitor to the subject. In some embodiments, the combination regimen comprises the steps of: (a) prescribing the subject to self-administer, and verifying that the subject has self-administered, the DNA-PK inhibitor prior to first administration of the PD-1 axis binding antagonist and TGFβ inhibitor; and (b) administering the PD-1 axis binding antagonist and TGFβ inhibitor to the subject. In some embodiments, the combination regimen comprises, after the subject has received the PD-1 axis binding antagonist and TGFβ inhibitor prior to the first administration of the DNA-PK inhibitor, administering the DNA-PK inhibitor to the subject. In some embodiments, the combination regimen comprises the steps of: (a) after the subject has received the PD-1 axis binding antagonist and TGFβ inhibitor prior to the first administration of the DNA-PK inhibitor, determining that an DNA-PK level in a cancer sample isolated from the subject exceeds an DNA-PK level predetermined prior to the first receipt of the PD-1 axis binding antagonist and TGFβ inhibitor, and (b) administering the DNA-PK inhibitor to the subject. In some embodiments, the combination regimen comprises, after the subject has received the DNA-PK inhibitor prior to first administration of the PD-1 axis binding antagonist and TGFβ inhibitor, administering the PD-1 axis binding antagonist and TGFβ inhibitor to the subject.

In some embodiments, the combination regimen comprises the steps of: (a) under the direction or control of a physician, the subject receiving the PD-1 axis binding antagonist and DNA-PK inhibitor prior to first receipt of the TGFβ inhibitor; and (b) under the direction or control of a physician, the subject receiving the TGFβ inhibitor. In some embodiments, the combination regimen comprises the steps of: (a) under the direction or control of a physician, the subject receiving the TGFβ inhibitor prior to first receipt of the PD-1 axis binding antagonist and DNA-PK inhibitor; and (b) under the direction or control of a physician, the subject receiving the PD-1 axis binding antagonist and DNA-PK inhibitor. In some embodiments, the combination regimen comprises the steps of: (a) prescribing the subject to self-administer, and verifying that the subject has self-administered, the PD-1 axis binding antagonist and DNA-PK inhibitor prior to first administration of the TGFβ inhibitor; and (b) administering the TGFβ inhibitor to the subject. In some embodiments, the combination regimen comprises the steps of: (a) prescribing the subject to self-administer, and verifying that the subject has self-administered, the TGFβ inhibitor prior to first administration of the PD-1 axis binding antagonist and DNA-PK inhibitor; and (b) administering the PD-1 axis binding antagonist and DNA-PK inhibitor to the subject. In some embodiments, the combination regimen comprises, after the subject has received the PD-1 axis binding antagonist and DNA-PK inhibitor prior to the first administration of the TGFβ inhibitor, administering the TGFβ inhibitor to the subject. In some embodiments, the combination regimen comprises, after the subject has received the TGFβ inhibitor prior to first administration of the PD-1 axis binding antagonist and DNA-PK inhibitor, administering the PD-1 axis binding antagonist and DNA-PK inhibitor to the subject.

Also provided herein is a PD-1 axis binding antagonist for use as a medicament in combination with a DNA-PK inhibitor and a TGFβ inhibitor. Similarly provided is a DNA-PK inhibitor for use as a medicament in combination with a PD-1 axis binding antagonist and a TGFβ inhibitor. Similarly provided is a TGFβ inhibitor for use as a medicament in combination with a PD-1 axis binding antagonist and a DNA-PK inhibitor. Similarly provided is an anti-PD-L1/TGFβ Trap for use as a medicament in combination with a DNA-PK inhibitor. Similarly provided is a combination of a TGFβ inhibitor, a PD-1 axis binding antagonist and a DNA-PK inhibitor for use as a medicament. Also provided is a PD-1 axis binding antagonist for use in the treatment of cancer in combination with a DNA-PK inhibitor and TGFβ inhibitor. Similarly provided is a DNA-PK inhibitor for use in the treatment of cancer in combination with a PD-1 axis binding antagonist and a TGFβ inhibitor. Similarly provided is a TGFβ inhibitor for use in the treatment of cancer in combination with a PD-1 axis binding antagonist and a DNA-PK inhibitor. Similarly provided is an anti-PD-L1/TGFβ Trap for use in the treatment of cancer in combination with a DNA-PK inhibitor. Similarly provided is a combination of a TGFβ inhibitor, a PD-1 axis binding antagonist and a DNA-PK inhibitor for use in the treatment of cancer.

Also provided is a combination comprising a PD-1 axis binding antagonist, a TGFβ inhibitor and a DNA-PK inhibitor. Also provided is a combination comprising a PD-1 axis binding antagonist, a TGFβ inhibitor and a DNA-PK inhibitor for use as a medicament. Also provided is a combination comprising a PD-1 axis binding antagonist, a TGFβ inhibitor and a DNA-PK inhibitor for the use in the treatment of cancer.

It shall be understood that, in the various embodiments described above, the PD-1 axis binding antagonist and the TGFβ inhibitor are preferably fused and, more preferably, correspond to anti-PD-L1/TGFβ Trap.

Also provided is the use of a combination for the manufacture of a medicament for the treatment of cancer, comprising a PD-1 axis binding antagonist, a TGFβ inhibitor and a DNA-PK inhibitor, wherein the anti-PD-L1 antibody preferably comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

The prior teaching of the present specification concerning the therapeutic combination, including the methods of using it, and all aspects and embodiments thereof, of this Section titled “Therapeutic combination and method of use thereof” is valid and applicable without restrictions to the medicament, the PD-1 axis binding antagonist, TGFβ inhibitor and/or DNA-PK inhibitor for use in the treatment of cancer as well as the combination, and aspects and embodiments thereof, of this Section, if appropriate.

Pharmaceutical Formulations and Kits

In some embodiments, the present invention provides a pharmaceutically acceptable composition comprising a PD-1 axis binding antagonist. In some embodiments, the present invention provides a pharmaceutically acceptable composition comprising a TGFβ inhibitor. In some embodiments, the present invention provides a pharmaceutically acceptable composition comprising anti-PD-L1/TGFβ Trap. In some embodiments, the present invention provides a pharmaceutically acceptable composition comprising a DNA-PK inhibitor, preferably Compound 1, or a pharmaceutically acceptable salt thereof. In some embodiments, the present invention provides a pharmaceutically acceptable composition of a chemotherapeutic agent. In some embodiments, the present invention provides a pharmaceutical composition comprising a PD-1 axis binding antagonist, a TGFβ inhibitor and at least a pharmaceutically acceptable excipient or adjuvant. In some embodiments, the present invention provides a pharmaceutical composition comprising a TGFβ inhibitor, a DNA-PK inhibitor and at least a pharmaceutically acceptable excipient or adjuvant. In some embodiments, the present invention provides a pharmaceutical composition comprising a PD-1 axis binding antagonist, a DNA-PK inhibitor and at least a pharmaceutically acceptable excipient or adjuvant. In some embodiments, the present invention provides a pharmaceutical composition comprising a PD-1 axis binding antagonist, a TGFβ inhibitor, a DNA-PK inhibitor and at least a pharmaceutically acceptable excipient or adjuvant. In the various embodiments described above and below, the anti-PD-L1 antibody preferably comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6 and, more preferably, is fused to the TGFβ inhibitor. In some embodiments, a composition comprising a DNA-PK inhibitor, preferably Compound 1, or a pharmaceutically acceptable salt thereof, is separate from a composition comprising a PD-1 axis binding antagonist, a TGFβ inhibitor and/or a chemotherapeutic agent. In some embodiments, a DNA-PK inhibitor, preferably Compound 1, or a pharmaceutically acceptable salt thereof, and a PD-1 axis binding antagonist, a TGFβ inhibitor and/or a chemotherapeutic agent are present in the same composition.

In some embodiments, a composition comprising the fused PD-1 axis binding antagonist and TGFβ inhibitor is separate from a composition comprising a DNA-PK inhibitor, preferably Compound 1, or a pharmaceutically acceptable salt thereof, and/or a chemotherapeutic agent. In some embodiments, a PD-1 axis binding antagonist and TGFβ inhibitor are fused and present with a DNA-PK inhibitor, preferably Compound 1, or a pharmaceutically acceptable salt thereof, and/or a chemotherapeutic agent in the same composition.

In certain embodiments, the present invention provides a composition comprising a DNA-PK inhibitor, preferably Compound 1, or a pharmaceutically acceptable salt thereof, and at least one of etoposide and cisplatin, optionally together with the PD-1 axis binding antagonist and/or TGFβ inhibitor. In some embodiments, a provided composition comprising a DNA-PK inhibitor, preferably Compound 1, or a pharmaceutically acceptable salt thereof, and at least one of etoposide and cisplatin is formulated for oral administration.

Examples of such pharmaceutically acceptable compositions are described further below and herein.

Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Liquid dosage forms for oral administration include, but are not limited to, pharmaceutically acceptable emulsions, microemulsions, solutions, suspensions, syrups and elixirs. In addition to Compound 1, or a pharmaceutically acceptable salt thereof, and/or a chemotherapeutic agent, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, lavouring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the PD-1 axis binding antagonist, TGFβ inhibitor, DNA-PK inhibitor, preferably Compound 1, and/or an additional chemotherapeutic agent, it is often desirable to slow absorption from subcutaneous or intramuscular injection. This may be accomplished by the use of a liquid suspension of crystalline or amorphous material with poor water solubility. The rate of absorption then depends upon its rate of dissolution that, in turn, may depend upon crystal size and crystalline form. Alternatively, delayed absorption of parenterally administered PD-1 axis binding antagonist, TGFβ inhibitor, DNA-PK inhibitor, preferably Compound 1, or a pharmaceutically acceptable salt thereof, and/or a chemotherapeutic agent, is accomplished by dissolving or suspending the compound in an oil vehicle. Injectable depot forms are made by forming microencapsule matrices of PD-1 axis binding antagonist, TGFβ inhibitor, DNA-PK inhibitor, preferably Compound 1, or a pharmaceutically acceptable salt thereof, and/or a chemotherapeutic agent, in biodegradable

polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound
in liposomes or microemulsions that are compatible with body tissues.

Compositions for rectal or vaginal administration are preferably suppositories, which can be prepared by mixing the compounds of this invention with suitable non-irritating excipients or carriers such as cocoa butter, polyethylene glycol or a suppository wax, which are solid at ambient temperature but liquid at body temperature and therefore melt in the rectum or vaginal cavity and release the active compound.

Dosage forms for oral administration include capsules, tablets, pills, powders, and granules, aqueous suspensions or solutions. In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidinone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The PD-1 axis binding antagonist, TGFβ inhibitor, DNA-PK inhibitor, preferably Compound 1, or a pharmaceutically acceptable salt thereof, and/or a chemotherapeutic agent, can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the PD-1 axis binding antagonist, TGFβ inhibitor, DNA-PK inhibitor, preferably Compound 1, or a pharmaceutically acceptable salt thereof, and/or a chemotherapeutic agent, may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the PD-1 axis binding antagonist, TGFβ inhibitor, DNA-PK inhibitor, preferably Compound 1, or a pharmaceutically acceptable salt thereof, and/or a chemotherapeutic agent, include ointments, pastes, creams, lotions, gels, powders, solutions, sprays, inhalants or patches. The active component is admixed under sterile conditions with a pharmaceutically acceptable carrier and any needed preservatives or buffers as may be required. Exemplary carriers for topical administration of compounds of this aremineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water. Ophthalmic formulation, ear drops, and eye drops are also contemplated as being within the scope of this invention.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Typically, the PD-1 axis binding antagonist or TGFβ inhibitor is incorporated into pharmaceutical compositions suitable for administration to a subject, wherein the pharmaceutical composition comprises the PD-1 axis binding antagonist or TGFβ inhibitor and a pharmaceutically acceptable carrier. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the PD-1 axis binding antagonist or TGFβ inhibitor.

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes, and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, or intramuscular). In a preferred embodiment, the PD-1 axis binding antagonist or TGFβ inhibitor is administered by intravenous infusion or injection. In another preferred embodiment, the PD-1 axis binding antagonist or TGFβ inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active PD-1 axis binding antagonist or TGFβ inhibitor in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, monostearate salts and gelatin.

In one embodiment, avelumab is a sterile, clear, and colorless solution intended for IV administration. The contents of the avelumab vials are non-pyrogenic, and do not contain bacteriostatic preservatives. Avelumab is formulated as a 20 mg/mL solution and is supplied in single-use glass vials, stoppered with a rubber septum and sealed with an aluminum polypropylene flip-off seal. For administration purposes, avelumab must be diluted with 0.9% sodium chloride (normal saline solution). Tubing with in-line, low protein binding 0.2 micron filter made of polyether sulfone (PES) is used during administration.

In a further aspect, the invention relates to a kit comprising a PD-1 axis binding antagonist and a package insert comprising instructions for using the PD-1 axis binding antagonist in combination with an DNA-PK inhibitor and a TGFβ inhibitor to treat or delay progression of a cancer in a subject. Also provided is a kit comprising an DNA-PK inhibitor and a package insert comprising instructions for using the DNA-PK inhibitor in combination with a PD-1 axis binding antagonist and a TGFβ inhibitor to treat or delay progression of a cancer in a subject. Also provided is a kit comprising a TGFβ inhibitor and a package insert comprising instructions for using the TGFβ inhibitor in combination with a PD-1 axis binding antagonist and an DNA-PK inhibitor to treat or delay progression of a cancer in a subject. Also provided is a kit comprising anti-PD-L1/TGFβ Trap and a package insert comprising instructions for using the anti-PD-L1/TGFβ Trap in combination with an DNA-PK inhibitor to treat or delay progression of a cancer in a subject. Also provided is a kit comprising a PD-1 axis binding antagonist and an DNA-PK inhibitor, and a package insert comprising instructions for using the PD-1 axis binding antagonist and the DNA-PK inhibitor in combination with a TGFβ inhibitor to treat or delay progression of a cancer in a subject. Also provided is a kit comprising a TGFβ inhibitor and an DNA-PK inhibitor, and a package insert comprising instructions for using the TGFβ inhibitor and the DNA-PK inhibitor in combination with a PD-1 axis binding antagonist to treat or delay progression of a cancer in a subject. Also provided is a kit comprising a PD-1 axis binding antagonist and a TGFβ inhibitor, and a package insert comprising instructions for using the PD-1 axis binding antagonist and the TGFβ inhibitor in combination with an DNA-PK inhibitor to treat or delay progression of a cancer in a subject. Also provided is a kit comprising anti-PD-L1/TGFβ Trap and an DNA-PK inhibitor, and a package insert comprising instructions for using anti-PD-L1/TGFβ Trap and the DNA-PK inhibitor to treat or delay progression of a cancer in a subject. The kit can comprise a first container, a second container, a third container and a package insert, wherein the first container comprises at least one dose of a medicament comprising the PD-1 axis binding antagonist, the second container comprises at least one dose of a medicament comprising the DNA-PK inhibitor, the third container comprises at least one dose of a medicament comprising the TGFβ inhibitor and the package insert comprises instructions for treating a subject for cancer using the medicaments. The first, second and third containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1, e.g., by means of an immunohistochemical (IHC) assay, FACS or LC/MS/MS.

The prior teaching of the present specification concerning the therapeutic combination, including the methods of using it, and all aspects and embodiments thereof, of the previous Section titled “Therapeutic combination and method of use thereof” is valid and applicable without restrictions to the pharmaceutical formulations and kits, and aspects and embodiments thereof, of this Section titled “Pharmaceutical formulations and kits”, if appropriate.

Further Diagnostic, Predictive, Prognostic and/or Therapeutic Methods

The disclosure further provides diagnostic, predictive, prognostic and/or therapeutic methods, which are based, at least in part, on determination of the identity of the expression level of a marker of interest. In particular, the amount of human PD-L1 in a cancer patient sample can be used to predict whether the patient is likely to respond favorably to cancer therapy utilizing the therapeutic combination of the invention. In some embodiments, the amount of human TGFβ in a cancer patient sample, preferably a serum sample, can be used to predict whether the patient is likely to respond favorably to cancer therapy utilizing the therapeutic combination of the invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the PD-L1 or TGFβ protein, DNA, RNA, or other suitable read-outs for PD-L1 or TGFβ levels, examples of which are described herein and/or are well known to the skilled artisan.

In some embodiments, determining the PD-L1 or TGFβ level comprises determining the PD-L1 or TGFβ expression. In some preferred embodiments, the PD-L1 or TGFβ level is determined by the PD-L1 or TGFβ protein concentration in a patient sample, e.g., with PD-L1 or TGFβ specific ligands, such as antibodies or specific binding partners. The binding event can, e.g., be detected by competitive or non-competitive methods, including the use of a labeled ligand or PD-L1 or TGFβ specific moieties, e.g., antibodies, or labeled competitive moieties, including a labeled PD-L1 or TGFβ standard, which compete with marker proteins for the binding event. If the marker specific ligand is capable of forming a complex with PD-L1 or TGFβ, the complex formation can indicate PD-L1 or TGFβ expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In a preferred embodiment, the PD-L1 or TGFβ expression is determined by immunohistochemistry using one or more primary anti-PD-L1 or anti-TGFβ antibodies.

In another embodiment, the biomarker RNA level is determined by a method comprising microarray chips, RT-PCR, qRT-PCR, multiplex qPCR or in-situ hybridization. In one embodiment of the invention, a DNA or RNA array comprises an arrangement of poly-nucleotides presented by or hybridizing to the PD-L1 or TGFβ gene immobilized on a solid surface. For example, to the extent of determining the PD-L1 or TGFβ mRNA, the mRNA of the sample can be isolated, if necessary, after adequate sample preparation steps, e.g., tissue homogenization, and hybridized with marker specific probes, in particular on a microarray platform with or without amplification, or primers for PCR-based detection methods, e.g., PCR extension labeling with probes specific for a portion of the marker mRNA.

Several approaches have been described for quantifying PD-L1 protein expression in IHC assays of tumor tissue sections (Thompson et al. (2004) PNAS 101(49): 17174; Thompson et al. (2006) Cancer Res. 66: 3381; Gadiot et al. (2012) Cancer 117: 2192; Taube et al. (2012) Sci Transl Med 4, 127ra37; and Toplian et al. (2012) New Eng. J Med. 366 (26): 2443). One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

The level of PD-L1 or TGFβ mRNA expression may be compared to the mRNA expression levels of one or more reference genes that are frequently used in quantitative RT-PCR, such as ubiquitin C. In some embodiments, a level of PD-L1 or TGFβ expression (protein and/or mRNA) by malignant cells and/or by infiltrating immune cells within a tumor is determined to be “overexpressed” or “elevated” based on comparison with the level of PD-L1 or TGFβ expression (protein and/or mRNA) by an appropriate control. For example, a control PD-L1 or TGFβ protein or mRNA expression level may be the level quantified in non-malignant cells of the same type or in a section from a matched normal tissue.

In a preferred embodiment, the efficacy of the therapeutic combination of the invention is predicted by means of PD-L1 or TGFβ expression in tumor samples.

Immunohistochemistry with anti-PD-L1 or anti-TGFβ primary antibodies can be performed on serial cuts of formalin fixed and paraffin embedded specimens from patients treated with an anti-PD-L1 antibody, such as avelumab, or an anti-TGFβ antibody.

This disclosure also provides a kit for determining if the combination of the invention is suitable for therapeutic treatment of a cancer patient, comprising means for determining a protein level of PD-L1 or TGFβ, or the expression level of its RNA, in a sample isolated from the patient and instructions for use. In another aspect, the kit further comprises avelumab for immunotherapy. In one aspect of the invention, the determination of a high PD-L1 or TGFβ level indicates increased PFS or OS when the patient is treated with the therapeutic combination of the invention. In one embodiment of the kit, the means for determining the PD-L1 or TGFβ protein level are antibodies with specific binding to PD-L1 or TGFβ, respectively.

In still another aspect, the invention provides a method for advertising a PD-1 axis binding antagonist in combination with a TGFβ inhibitor and an DNA-PK inhibitor, comprising promoting, to a target audience, the use of the combination for treating a subject with a cancer based on PD-L1 and/or TGFβ expression in samples taken from the subject. In still another aspect, the invention provides a method for advertising an DNA-PK inhibitor in combination with a PD-1 axis binding antagonist and a TGFβ inhibitor, which are preferably fused, comprising promoting, to a target audience, the use of the combination for treating a subject with a cancer based on PD-L1 and/or TGFβ expression in samples taken from the subject. In still another aspect, the invention provides a method for advertising a TGFβ inhibitor in combination with a PD-1 axis binding antagonist and an DNA-PK inhibitor, comprising promoting, to a target audience, the use of the combination for treating a subject with a cancer based on PD-L1 and/or TGFβ expression in samples taken from the subject. In still another aspect, the invention provides a method for advertising a combination comprising a PD-1 axis binding antagonist, a TGFβ inhibitor and an DNA-PK inhibitor, comprising promoting, to a target audience, the use of the combination for treating a subject with a cancer based on PD-L1 and/or TGFβ expression in samples taken from the subject. Promotion may be conducted by any means available. In some embodiments, the promotion is by a package insert accompanying a commercial formulation of the therapeutic combination of the invention. The promotion may also be by a package insert accompanying a commercial formulation of the PD-1 axis binding antagonist, TGFβ inhibitor, DNA-PK inhibitor or another medicament (when treatment is a therapy with the therapeutic combination of the invention and a further medicament). Promotion may be by written or oral communication to a physician or health care provider. In some embodiments, the promotion is by a package insert where the package insert provides instructions to receive therapy with the therapeutic combination of the invention after measuring PD-L1 and/or TGFβ expression levels, and in some embodiments, in combination with another medicament. In some embodiments, the promotion is followed by the treatment of the patient with the therapeutic combination of the invention with or without another medicament. In some embodiments, the package insert indicates that the therapeutic combination of the invention is to be used to treat the patient if the patient's cancer sample is characterized by high PD-L1 and/or TGFβ biomarker levels. In some embodiments, the package insert indicates that the therapeutic combination of the invention is not to be used to treat the patient if the patient's cancer sample expresses low PD-L1 and/or TGFβ biomarker levels. In some embodiments, a high PD-L1 and/or TGFβ biomarker level means a measured PD-L1 and/or TGFβ level that correlates with a likelihood of increased PFS and/or OS when the patient is treated with the therapeutic combination of the invention, and vice versa. In some embodiments, the PFS and/or OS is decreased relative to a patient who is not treated with the therapeutic combination of the invention. In some embodiments, the promotion is by a package insert where the package inset provides instructions to receive therapy with anti-PD-L1/TGFβ Trap in combination with an DNA-PK inhibitor after first measuring PD-L1 and/or TGFβ. In some embodiments, the promotion is followed by the treatment of the patient with anti-PD-L1/TGFβ Trap in combination with an DNA-PK inhibitor with or without another medicament. Further methods of advertising and instructing, or business methods applicable in accordance with the invention are described (for other drugs and biomarkers) in US 2012/0089541, for example.

The prior teaching of the present specification concerning the therapeutic combination, including the methods of using it, and all aspects and embodiments thereof, of the previous Section titled “Therapeutic combination and method of use thereof” is valid and applicable without restrictions to the methods and kits, and aspects and embodiments thereof, of this Section titled “Further diagnostic, predictive, prognostic and/or therapeutic methods”, if appropriate.

All the references cited herein are incorporated by reference in the disclosure of the invention hereby.

It is to be understood that this invention is not limited to the particular molecules, pharmaceutical compositions, uses and methods described herein, as such matter can, of course, vary. It is also to be understood that the terminology used herein is for the purpose of describing particular embodiments only and is not intended to limit the scope of the present invention, which is only defined by the appended claims. The techniques that are essential according to the invention are described in detail in the specification. Other techniques which are not described in detail correspond to known standard methods that are well known to a person skilled in the art, or the techniques are described in more detail in cited references, patent applications or standard literature.

Provided that no other hints in the application are given, they are used as examples only, they are not considered to be essential according to the invention, but they can be replaced by other suitable tools and biological materials.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### EXAMPLES

### Example 1: DNA-PK Inhibitor in Combination with Avelumab

The combination potential of M3814 (Compound 1) and Avelumab was elaborated in mice using the murine colon tumor model MC38. This model allows the use of immunocompetent mice, a necessary requirement to study the T-cell mediated antitumor effect of Avelumab. The experimental set up included the induction of MC38 tumors in C57BL6/N mice by injection of 1×10⁶ tumor cells into the right flank of the animals. Tumor growth was followed over time by measuring length and width using a caliper. When tumors were established to an average size of 50-100 mm³, mice were subdivided in 4 treatment groups with 10 animals each, and treatment started. This day was defined as day 0. Group 1 received vehicle treatment. Group 2 received M3814 orally once daily at 150 mg/kg in a volume of 10 ml/kg. Group 3 received avelumab intravenously once daily at 400 μg/mouse in a volume of 5 ml/kg on days 3, 6 and 9. Group 4 received M3814 orally once daily at 150 mg/kg in a volume of 10 ml/kg and avelumab intravenously once daily at 400 μg/mouse in a volume of 5 ml/kg on days 3, 6 and 9.

As a result of the study, the combined treatment of M3814 and avelumab was significantly superior to either of the monotherapy treatments (FIG. 3). A Kaplan-Meyer evaluation of the data revealed that the median time the tumors of the respective treatment groups needed to double in size as compared to their initial volume at day 0 was 6 days for Group 1, 10 days for Group 2, 13 days for Group 3, and 20 days for group 4. The respective T/C values calculated at day 13 were 47% for Group 2, 60% for Group 3, and 21% for Group 4. The treatment was overall well tolerated.

### Example 2: DNA-PK Inhibitor in Combination with Avelumab and Radiotherapy

The combination potential of M3814 (Compound 1), avelumab and radiotherapy was elaborated in mice using the murine colon tumor model MC38. This model allows the use of immunocompetent mice, a necessary requirement to study the T-cell mediated antitumor effect of avelumab. The experimental set up included the induction of MC38 tumors in C57BL6/N mice by injection of 1×10⁶ tumor cells into the right flank of the animals. Tumor growth was followed over time by measuring length and width using a caliper. When tumors were established to an average size of 50-100 mm³, mice were subdivided in 4 treatment groups with 10 animals each, and treatment started. This day was defined as day 0. Group 1 received Ionizing radiation (IR) at a daily dose of 2 Gy for 5 consecutive days and vehicle treatment. Group 2 received IR at a daily dose of 2 Gy for 5 consecutive days and M3814 orally once daily at 100 mg/kg in a volume of 10 ml/kg for 5 consecutive days, 30 min prior to each IR fraction. Group 3 received IR at a daily dose of 2 Gy for 5 consecutive days and avelumab intravenously once daily at 400 μg/mouse in a volume of 5 ml/kg on days 8, 11 and 14. Group 4 received IR at a daily dose of 2 Gy for 5 consecutive days and M3814 orally once daily at 100 mg/kg in a volume of 10 ml/kg for 5 consecutive days, 30 min prior to each IR fraction and avelumab intravenously once daily at 400 μg/mouse in a volume of 5 ml/kg on days 8, 11 and 14.

As a result of the study the combined treatment of M3814, avelumab and IR was significantly superior to M3814 and IR as well as avelumab and IR (FIG. 4). A Kaplan-Meyer evaluation of the data revealed that the median time the tumors of the respective treatment groups needed to double in size as compared to their initial volume at day 0 was 10 days for Group 1, 21 days for Group 2, 10 days for Group 3, and not reached for Group 4 by study end on day 28 because 60% of the animals did not reach the respective tumor volume. The treatment was overall well tolerated.

### Example 3: DNA-PK Inhibitor in Combination with Anti-PD-L1/TGFβ Trap and Radiotherapy

### Example 3A: Triple Combination with Anti-PD-L1/TGFβ Trap, Radiation Therapy, and M3814 Enhanced Antitumor Activity in a Mouse Mammary Tumor Model

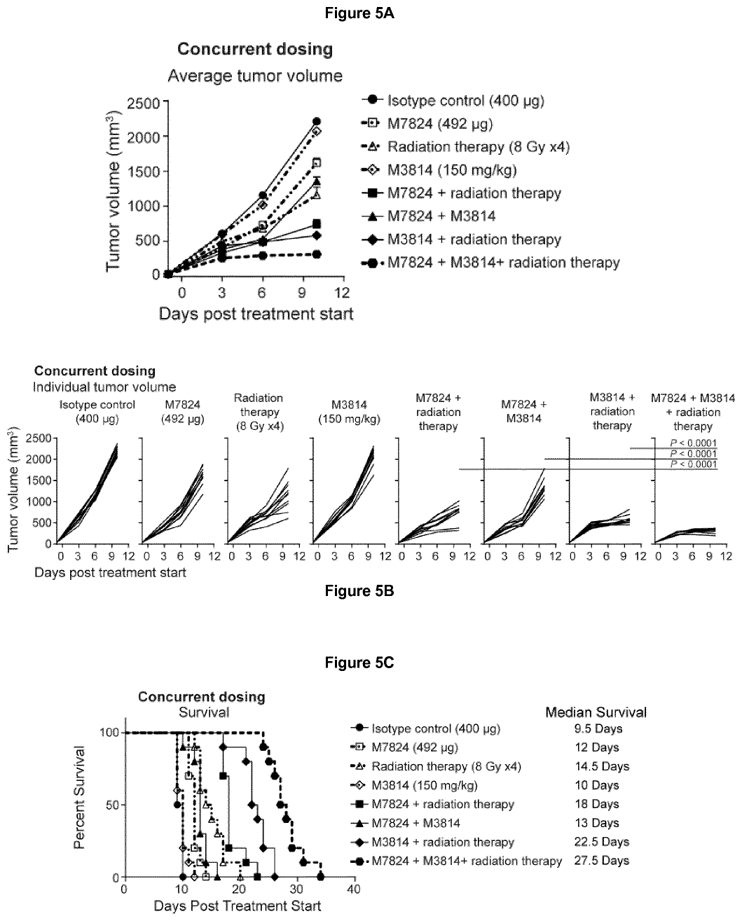

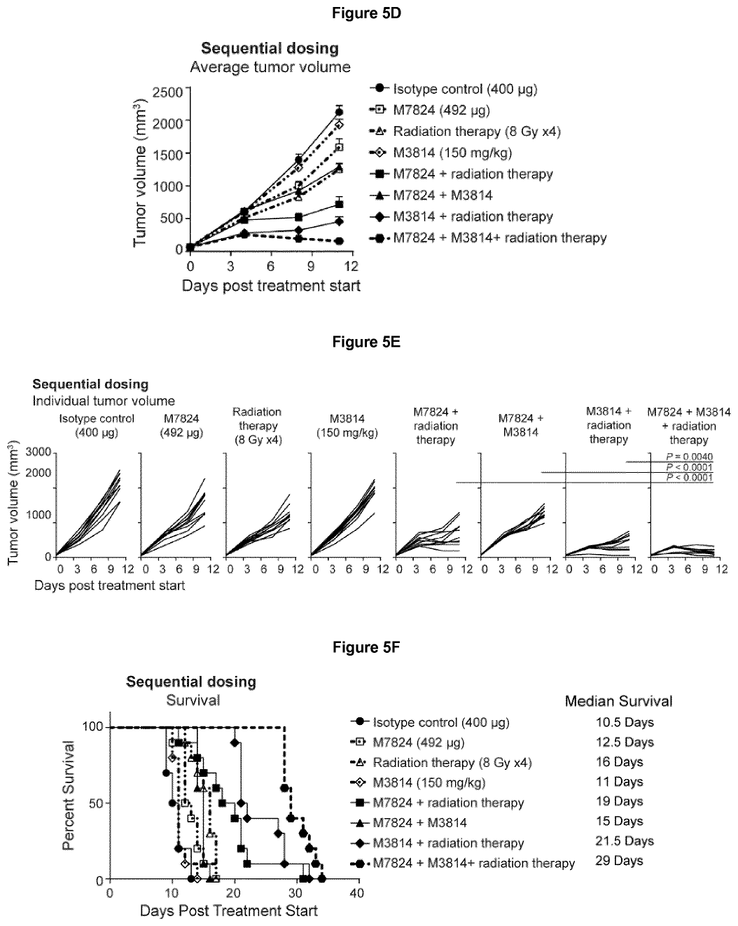

The anti-tumor efficacy of triple combination therapy with anti-PD-L1/TGFβ Trap (also referred to as M7824 in the Figures), M3814 (Compound 1), and radiation therapy was evaluated in Balb/C mice bearing 4T1 mammary tumors when anti-PD-L1/TGFβ Trap (492 μg; day 0, 2, 4) and radiation therapy (8 Gy, day 0-3) were administered concurrently. Monotherapy with anti-PD-L1/TGFβ Trap or radiation therapy significantly decreased tumor volume relative to isotype control (P<0.0001 and P<0.0001, respectively, day 10). In contrast, M3814 monotherapy did not significantly affect tumor growth (P=0.1603, day 10). Combination of M3814 with radiation therapy, however, significantly decreased tumor volume relative to M3814 or radiation alone (P<0.0001 and P<0.0001, respectively, day 10), and combination of M3814 with anti-PD-L1/TGFβ Trap significantly decreased tumor volume relative to M3814 or radiation alone (P<0.0001 and P<0.0001, respectively, day 10) (FIG. 5, A-B), suggesting that M3814 synergizes with radiation therapy or anti-PD-L1/TGFβ Trap to elicit enhance antitumor efficacy. Combining radiation with anti-PD-L1/TGFβ Trap resulted in similarly enhanced tumor growth inhibition relative to either radiation or anti-PD-L1/TGFβ Trap alone (P<0.0001 and P<0.0001, respectively, day 10) (FIG. 5, A-B). With triple combination therapy, tumor volume was further decreased relative to any of the dual therapy combinations (P<0.0001 for all, day 10) (FIG. 5, A-B). In addition, survival was extended with triple combination therapy to a greater degree than any other therapy; median survival was 27.5 days compared with 22.5 days for dual combination with radiation and M3814 (P=0.0002), 18 days for dual combination with anti-PD-L1/TGFβ Trap and radiation (P<0.0001), and 13 days for dual combination with anti-PD-L1/TGFβ Trap and M3814 (P<0.0001) (FIG. 5C).

The anti-tumor efficacy of triple combination therapy was also evaluated in Balb/C mice bearing 4T1 mammary tumors when anti-PD-L1/TGFβ Trap (492 μg; day 4, 6, 8) and radiation therapy (8 Gy, day 0-3) were administered sequentially. Similar to results for concurrent dosing, when anti-PD-L1/TGFβ Trap was dosed following radiation therapy, the monotherapies decreased tumor volume relative to isotype control (P<0.0001 and P<0.0001, respectively, day 11) and triple combination therapy further decreased tumor volume relative to dual therapy with anti-PD-L1/TGFβ Trap and radiation (P=0.0040, day 11), anti-PD-L1/TGFβ Trap and M3814 (P<0.0001, day 11), or M3814 and radiation (P<0.0001, day 11) (FIG. 5, D-E). Survival was also extended with triple combination therapy to a greater degree than any other therapy; median survival was 29 days compared with dual combination with anti-PD-L1/TGFβ Trap and radiation (19 days, P=0.0005), anti-PD-L1/TGFβ Trap and M3814 (15 days, P<0.0001), or M3814 and radiation (21.5 days, P=0.0019) (FIG. 5F). Taken together, these findings demonstrate that triple combination treatment with anti-PD-L1/TGFβ Trap, M3814, and radiation enhanced anti-tumor activity relative to dual combinations or monotherapies in the 4T1 model, regardless of whether the dosing schedule was concurrent or sequential.

### Example 3B: Triple Combination with Anti-PD-L1/TGFβ Trap, Radiation Therapy, and M3814 Enhanced Antitumor Activity in a Mouse Glioblastoma (GBM) Mouse Tumor Model

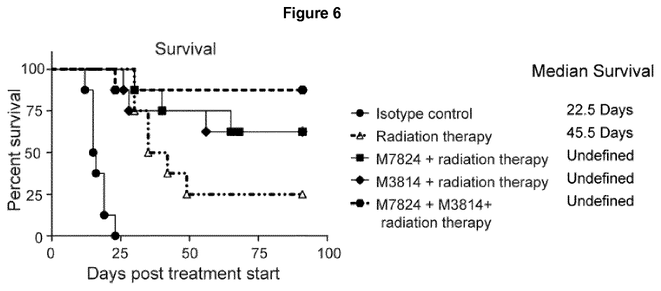

The GL261 glioblastoma (GBM) mouse model has been widely used for preclinical testing of immunotherapeutics for GBM, but is moderately immunogenic and known to evade host immune recognition. Therefore, the GL261 tumor model was used to evaluate whether adding anti-PD-L1/TGFβ Trap and/or M3814 treatment could improve the effects of radiation therapy, part of the standard treatment for patients with GBM. Triple combination therapy with anti-PD-L1/TGFβ Trap, radiation, and M3814 extended survival to a greater degree than radiation therapy alone (P=0.0248), whereas dual combination with anti-PD-L1/TGFβ Trap and radiation (P=0.1136) or anti-PD-L1/TGFβ Trap and radiation (P=0.1992) had no significantly extended survival relative to radiation alone (FIG. 6).

### Example 3C: Triple Combination with Anti-PD-L1/TGFβ Trap, Radiation Therapy, and M3814 Enhanced Antitumor Activity in a the MC38 Colorectal Carcinoma Model

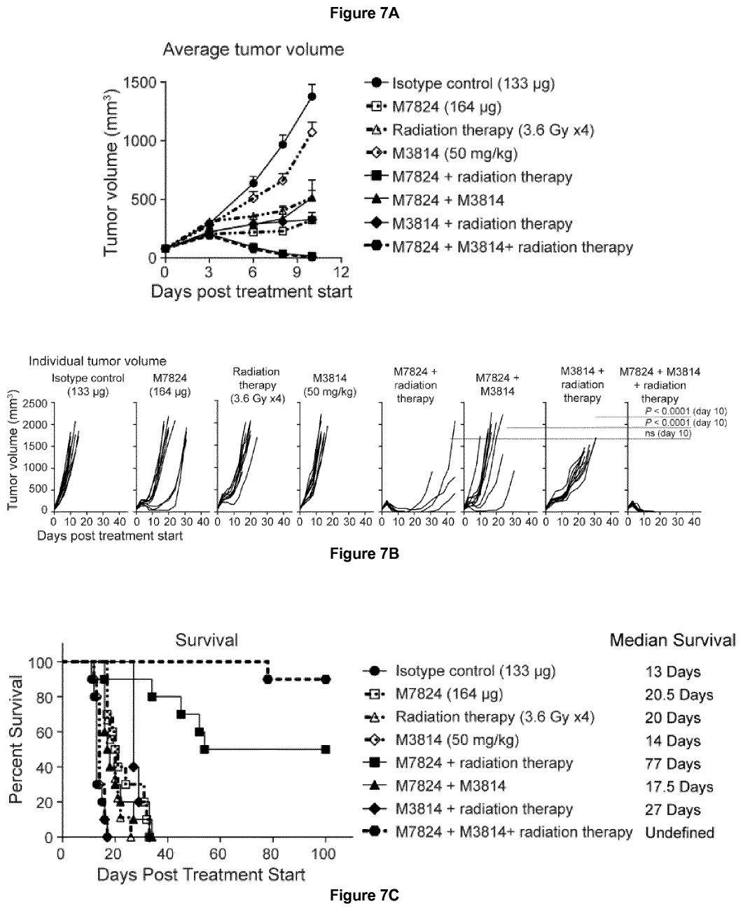

In the MC38 colorectal carcinoma model, dual therapies partially inhibited tumor growth. However, triple combination therapy with anti-PD-L1/TGFβ Trap, radiation therapy, and M3814 resulted in superior tumor regression relative to dual combination with anti-PD-L1/TGFβ Trap and M3814 (P>0.0001, day 10) and M3814 and radiation therapy (P>0.0001, day 10) (FIG. 7A-B). In fact, all mice (100%, 10 of 10 mice) treated with triple combination therapy had complete tumor regression over the duration of the experiment. In comparison, complete tumor regression was only observed in one other treatment group, anti-PD-L1/TGFβ Trap and radiation dual combination (56%, 5 of 9 mice), while the other treatment groups had no complete regressions (0%, 0 of 10 mice) (FIG. 7B). Triple combination therapy also extended survival to a greater degree than any other therapy. At the end of the experimental time course (100 days), 90% of mice were still alive in the triple combination group, which exceeded the median survival of dual combination with radiation and M3814 (27 days, P<0.0001), anti-PD-L1/TGFβ Trap and radiation (77 days, P=0.0406), and anti-PD-L1/TGFβ Trap and M3814 (17.5 days, P<0.0001) (FIG. 7C).

### Example 3D: Triple Combination with Anti-PD-L1/TGFβ Trap, Radiation Therapy, and M3814 Induced an Abscopal Effect in the MC38 Model

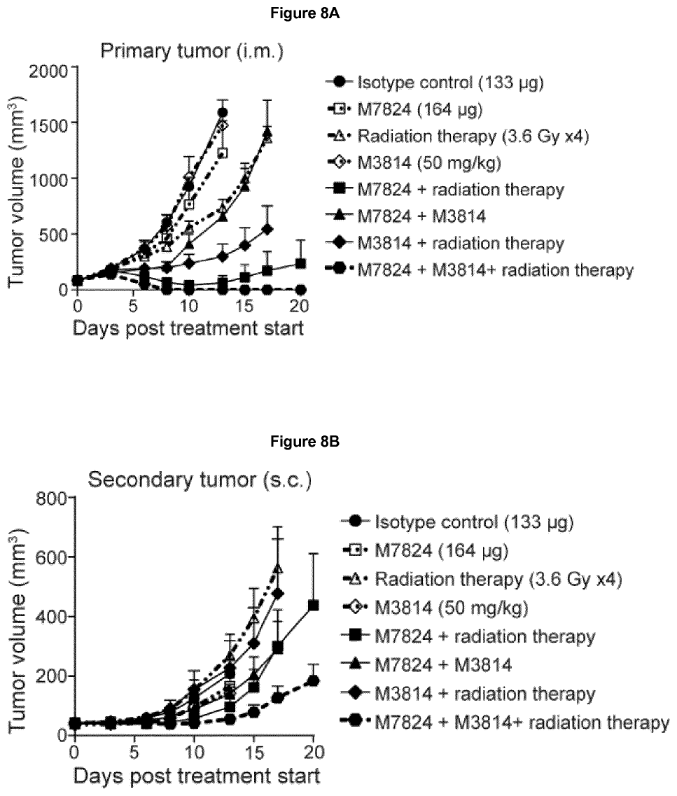

A study was conducted to test the potential abscopal effect of the triple combination therapy with anti-PD-L1/TGFβ Trap, radiation therapy, and M3814 in C57BL/6 mice bearing a primary i.m. MC38 tumor and a distal subcutaneous (s.c.) MC38 tumor. The localized fractionated radiation was applied to the primary tumor only. Similar to the 4T1 and GL261-Luc2 models, triple combination therapy significantly reduced tumor growth in the primary tumor, even relative to anti-PD-L1/TGFβ Trap and radiation therapy (P=0.0006, day 20) (FIG. 8A). Triple combination therapy was also able to induce an abscopal effect and significantly reduce growth of the secondary tumor relative to the dual combination of anti-PD-L1/TGFβ Trap and radiation therapy (P=0.0072, day 20) (FIG. 8B).

### Example 3E: Triple Combination with Anti-PD-L1/TGFβ Trap, Radiation Therapy, and M3814 Induced an Abscopal Effect in the 4T1 Model

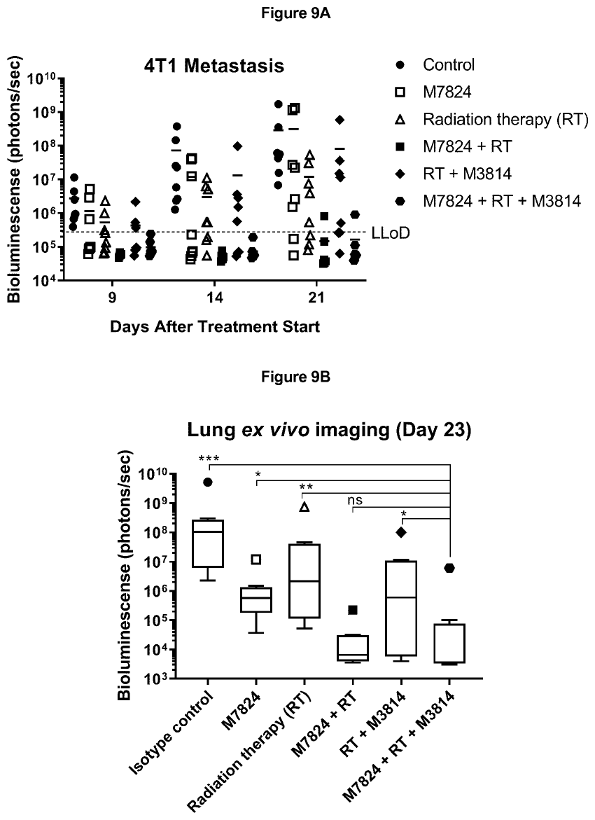

To test the potential abscopal effect of the triple combination therapy with anti-PD-L1/TGFβ Trap, radiation therapy, and M3814 in the 4T1 model, a luciferase-expressing 4T1 tumor cell line (4T1-Luc2-1A4) was injected orthotopically in BALB/c mice and spontaneous lung metastases were evaluated. Localized radiation was applied to the primary orthotopic tumor only via Small Animal Radiation Research Platform (SARRP) and in vivo and ex vivo lung metastases were visualized with bioluminescence imaging (BLI) on an IVIS Spectrum. In vivo imaging on days 9, 14, and 21 after treatment start showed that both anti-PD-L1/TGFβ Trap and radiation therapy dual combination therapy and anti-PD-L1/TGFβ Trap, radiation therapy, and M3814 triple combination therapy reduced mean BLI (a measure of lung metastases) below the lower level of detection (LLoD), whereas other treatment groups did not (FIG. 9A). At day 23, triple combination therapy significantly reduced BLI levels in ex vivo lungs relative isotype control (P=0.0006), anti-PD-L1/TGFβ Trap (P=0.0104), radiation therapy (P=0.0070), and radiation therapy+M3814 (P=0.0207), but not relative to anti-PD-L1/TGFβ Trap+radiation dual therapy (P=0.1605) (FIG. 9B). These results suggest that anti-PD-L1/TGFβ Trap and radiation therapy synergize to induce an abscopal effect in the 4T1 model.

### Example 3F: Triple Combination with Anti-PD-L1/TGFβ Trap, Radiation Therapy, and M3814 Increased CD8⁺ Tumor Infiltrating Lymphocytes (TILs) in the 4T1 Model

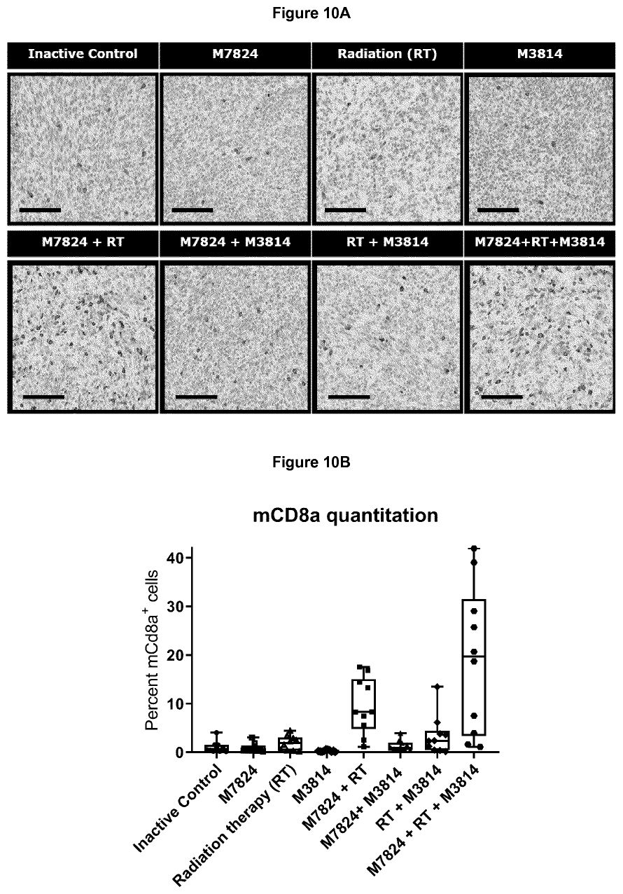

Immunohistochemistry (IHC) analysis of 4T1 tumor-bearing BALB/c mice revealed that the combination of anti-PD-L1/TGFβ Trap, radiation therapy, and M3814 resulted in an influx of CD8⁺ cells in the tumor 10 days after treatment start (FIG. 10A). Quantification of IHC images showed that triple combination therapy significantly increased the percentage of CD8⁺ tumor infiltrating lymphocytes (TILs) relative to anti-PD-L1/TGFβ Trap+radiation therapy (P=0.0045), anti-PD-L1/TGFβ Trap+M3814 (P<0.0001), and radiation+M3814 (P<0.0001) treatments (FIG. 10B). These results suggest that the combination of all three treatments, anti-PD-L1/TGFβ Trap, radiation therapy, and M3814, is necessary to induce the highest percentage of CD8+TILs.

### Example 3G: Triple Combination with Anti-PD-L1/TGFβ Trap, Radiation Therapy, and M3814 Induced Gene Expression Changes in EMT, Fibrosis, and VEGF Pathway Signatures

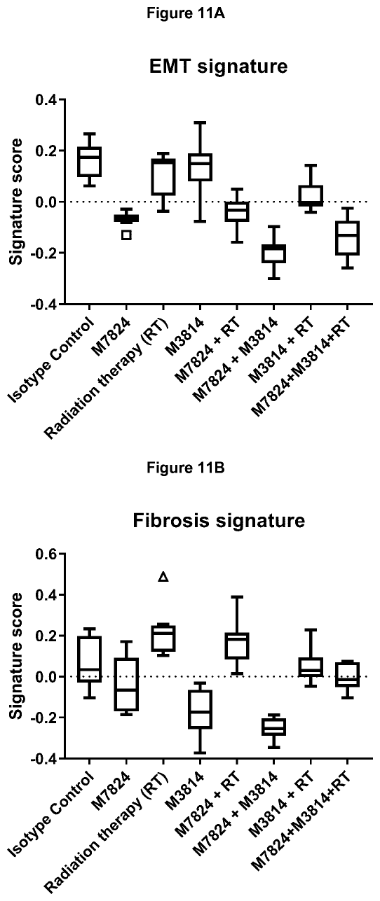

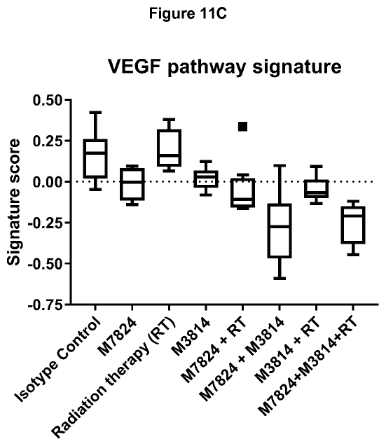

To evaluate the effects of anti-PD-L1/TGFβ Trap, radiation therapy, and M3814 treatment on the tumor microenvironment, 4T1 tumor tissue was analyzed by RNA sequencing (RNAseq) and gene signatures associated with EMT, fibrosis, and the VEGF pathway were evaluated. Anti-PD-L1/TGFβ Trap significantly reduced the EMT signature score relative to isotype control (P<0.0001), whereas radiation therapy alone had no significant effect (FIG. 11A). Although M3814 monotherapy also had no effect on the EMT signature, combination of anti-PD-L1/TGFβ Trap and M3814 significantly decreased the signature score relative to anti-PD-L1/TGFβ Trap monotherapy (P=0.0077), suggesting possible synergy in this dual combination (FIG. 11A). Triple combination treatment did not significantly decrease the EMT signature relative to anti-PD-L1/TGFβ Trap and M3814 combination or anti-PD-L1/TGFβ Trap and RT combination, but it did decrease EMT signature relative to radiation therapy and M3814 combination (FIG. 11A), suggesting that the effect was mainly driven by anti-PD-L1/TGFβ Trap, with potential synergy between anti-PD-L1/TGFβ Trap and M3814.

Radiation therapy slightly, though not significantly, increased the fibrosis signature score in 4T1 tumors (P=0.0550), while M3814 significantly decreased the score (P=0.0002) and anti-PD-L1/TGFβ Trap trended but did not have a significant decrease in the fibrosis signature (FIG. 11B). Combination with anti-PD-L1/TGFβ Trap and M3814 further decreased the fibrosis signature relative to anti-PD-L1/TGFβ Trap monotherapy (P=0.0007), but the addition of radiation therapy in the triple combination significantly increased fibrosis signature relative to the anti-PD-L1/TGFβ Trap and M3814 dual therapy (P<0.0001). However, the signature score of the triple combination was not significantly different from isotype control (FIG. 11B), suggesting that radiation therapy negates the decrease in expression of fibrosis-associated genes seen with M3814 and anti-PD-L1/TGFβ Trap combination treatment.

Finally, VEGF pathway signature scores were unaffected by any of the monotherapy treatments (FIG. 11C). However, anti-PD-L1/TGFβ Trap and M3814 dual combination significantly reduced this signature relative to isotype control (P<0.0001), anti-PD-L1/TGFβ Trap monotherapy (P=0.0037), and M3814 monotherapy (P=0.0004). Triple combination therapy did not affect the VEGF pathway signature relative to anti-PD-L1/TGFβ Trap and M3814 combination but reduced the score relative to M3814 and radiation combination (P=0.0287) and anti-PD-L1/TGFβ Trap and radiation combination (P=0.0217) (FIG. 11C). These results suggest that a decrease in VEGF pathway gene expression seen with the triple combination was mainly driven by a possible synergy between anti-PD-L1/TGFβ Trap and M3814.

### Materials and Methods of Examples 3A-G

Cell Lines

4T1 murine breast cancer cells were obtained from the American Type Culture Collection (ATCC). 4T1-Luc2-1A4 luciferase cells were obtained from Caliper/Xenogen. The GL261-Luc2 murine glioma cell line was from PE (Xenogen) (Caliper). The MC38 murine colon carcinoma cell line was a gift from the Scripps Research Institute. 4T1 cells were cultured in RPM11640 medium supplemented with 10% heat-inactivated fetal bovine serum (FBS) (Life Technologies) and 4T1-Luc2-1A4 cells were also cultured in RPM11640 media and implanted in serum-free media and 50% matrigel. GL261-Luc2 cells were cultured in Dulbecco's Modified Eagle Medium (DMEM) containing 10% FBS and 1× penicillin/streptomycin/L-glutamine. MC38 cells were cultured in DMEM containing 10% FBS (Life Technologies). All cells were cultured under aseptic conditions and incubated at 37° C. with 5% CO₂. Cells were passaged before in vivo implantation and adherent cells were harvested with TrypLE Express (Gibco) or 0.25% trypsin.

Mice

BALB/c, C57BL/6, and albino C57BL/6 mice were obtained from Charles River Laboratories, Jackson Laboratories, or Envigo, respectively. For abscopal experiments with 4T1-Luc2-1A4 cells, all studies were performed by Mi Bioresearch and BALB/c mice were obtained from Envigo. All mice used for experiments were 6- to 12-week-old females. All mice were housed with ad libitum access to food and water in pathogen-free facilities.

Murine Tumor Models

4T1 Tumor Model

For efficacy and survival studies, 4T1 cells, 0.5×10⁵, were inoculated intramuscularly (i.m.) in the thigh of BALB/c mice on day −6. Treatment was initiated 6 days later on day 0, and mice were sacrificed when tumor volumes reached ˜2000 mm³.

For abscopal experiments, 0.5×10⁶ 4 T1-Luc2-1A4 cells were inoculated orthotopically in the mammary fat pad in BALB/c mice on day −9. Treatment was initiated 9 days later on day 0, and mice were sacrificed on day 23 for ex vivo lung imaging.

For IHC studies, 4T1 cells, 0.5×10⁵, were inoculated intramuscularly (i.m.) in the thigh of BALB/c mice on day −7. Treatment was initiated 7 days later on day 0, and mice were sacrificed on day 10.

For RNAseq study, 4T1 cells, 0.5×10⁵, were inoculated intramuscularly (i.m.) in the thigh of BALB/c mice on day −6. Treatment was initiated 6 days later on day 0, and mice were sacrificed on day 6.

GL261 Tumor Model

For efficacy studies, GL261-Luc2 cells, 1×10⁶ in 10 μl, were implanted orthotopically via intracranial injection on day −7 in albino C57BL/6 females. All surgical procedures were conducted in compliance with all the laws, regulations, and guidelines of the National Institute of Health (NIH) and with the approval of MI Bioresearch's Animal Care and Use Committee (IACUC). Briefly, mice were dosed s.c. with 5 mg/kg Carprofen 30 minutes prior to surgery and anesthetized with 2% isoflurane in air during surgical implantation. Tumor cells were injected using a stereotaxic device with the coordinates, Bregma: 1 mm anterior, 2 mm right lateral, and 2 mm ventral into brain. A second dose of Carprofen was administered 24 hours post-surgery. Treatment was initiated on day 0, and, for survival analysis, mice were sacrificed when they reached a moribund state.

MC38 Tumor Model

For efficacy and survival studies, MC38 cells, 0.25×10⁶, were inoculated i.m. in the thigh of BALB/c mice on day −7. Treatment was initiated 7 days later on day 0, and mice were sacrificed when tumor volumes reached ˜2000 mm³.

For MC38 abscopal effect studies, 0.25×10⁶ MC38 cells were inoculated i.m. in the right thigh with a second distal s.c. inoculation of 1×10⁶ MC38 cells in the left flank on day −7. Treatment was initiated 7 days later on day 0.

Treatment

For all studies, mice were randomized into treatment groups on the day of treatment initiation (day 0).

Anti-PD-L1/TGFβ Trap and Isotype Control

Anti-PD-L1/TGFβ Trap is a full human immunoglobulin 1 (IgG1) monoclonal antibody against human PD-L1 fused to the extracellular domain of human TGF-3 receptor II. The isotype control is a mutated version of anti-PD-L1, which completely lacks PD-L1 binding. In tumor-bearing mice, anti-PD-L1/TGFβ Trap (164, 492 μg) or isotype control (133, 400 μg) were administered with an intravenous injection (i.v.) in 0.2 mL PBS. Exact dose and treatment schedules for each experiment are listed in the figure legends. Tumor-bearing mice were treated with 1-3 doses spaced 2 days apart for 1-4 days.

M3814 and Vehicle Control

M3814 is a selective DNA-PK inhibitor, and the vehicle is 0.25% Methocel® K4M Premium+0.25% Tween® 20 in Sodium (Na) Citrate Buffer 300 mM, pH 2.5. In tumor-bearing mice, M3814 (50, 150 mg/kg) or vehicle control (0.2 mL) were administered via oral gavage (p.o.). Exact dose and treatment schedules for each experiment are listed in the figure legends. Tumor-bearing mice were treated with 1 dose per day for 14 days.

Radiation

To assess the combination of radiation with anti-PD-L1/TGFβ Trap and/or M3814 mice were randomized into the following treatment groups: isotype control (133, 400 μg)+vehicle control (0.2 mL), radiation (3.6, 7.5, 8, 10 Gy/day), anti-PD-L1/TGFβ Trap (164, 492 μg), M3814 (50, 150 mg/kg), anti-PD-L1/TGFβ Trap+M3814, anti-PD-L1/TGFβ Trap+radiation, M3814+radiation, or anti-PD-L1/TGFβ Trap+M3814+radiation. All non-anti-PD-L1/TGFβ Trap groups received isotype control and all non-M3814 groups received vehicle control. To deliver radiation treatment to i.m. tumors, a collimator device with lead shielding was used to localize delivery to the tumor-bearing thigh of mice. This region was irradiated by timed exposure to a Cesium-137 gamma irradiator (GammaCell® 40 Exactor, MDS Nordion, Ottawa, ON, Canada). Radiation treatment was given once per day for four days. To deliver radiation to orthotopic mammary fat pad tumors, for 4T1 abscopal study, focal beam radiation treatment was administered via the Xstrahl Life Sciences Small Animal Radiation Research Platform (SAARP). This system allows for highly targeted irradiation which mimics that applied in human patients. SAARP irradiation is delivered using CT-guided targeting. Radiation treatment was given once on day 0.

For GL261 studies, radiation treatment was administered via the Xstrahl Life Sciences Small Animal Radiation Research Platform. Treatment (220 kV, 13.0 mA) was applied using a 10 mm collimator and delivered to a total dose of 7.5 Gy in 2 equally weighted beams. Radiation treatment was given once on day 0.

Tumor Growth and Survival

Tumor sizes for the 4T1 and MC38 models were measured twice per week with digital calipers and recorded automatically using WinWedge software. Tumor volumes were calculated with the following formula: tumor volume (mm³)=tumor length×width×height×0.5236. To compare the percentage survival between different treatment groups, Kaplan-Meier survival curves were generated; mice were sacrificed when their tumor volume exceeded ≈2,000 mm³. For the GL261 tumor model, mice were monitored for health and sacrificed when they reached a moribund state.

In Vivo and Ex Vivo Bioluminescence Imaging (BLI)

To acquire in vivo BLI images on Days 9, 14 and 21 post treatment start, D-Luciferin (Promega) was prepared at 15 mg/ml and each mouse was injected i.p. with 150 mg/kg 10 minutest prior to imaging and under 1-2% isoflurane gas anesthesia. BLI was performed using an IVIS Spectrum (PerkinElmer, MA). The primary tumor was shielded prior to imaging so that metastatic signal in the thoracic region could be quantified. Large binning of the CCD chip was used, and the exposure time was adjusted (10 seconds to 2 minutes) to obtain at least several hundred counts per image and to avoid saturation of the CCD chip. Images were analyzed using Living Image 4.3.1 (PerkinElmer, MA) software.

Ex vivo BLI was performed on all animals on Day 23. D-luciferin (150 mg/kg) was injected into mice 10 minutes before they were euthanized. Lungs were then harvested, weighed, and placed in D-luciferin (300 μg/ml in saline) in individual wells of 24-well black plates. All harvested tissues were then imaged over 2-3 minutes using large (high sensitivity) binning. Where necessary, tissue emitting very bright signals was removed or shielded in order to re-image the plate to potentially detect tissues with weaker signals.

Anti-CD8 Immunohistochemistry and Quantification

4T1 FFPE tumor sections (5 μm) mounted on SuperFrost® Plus slides were stained on the Leica Bond autostainer using established protocols. Briefly, slides were baked, dewaxed, rehydrated, and subjected to antigen retrieval for 20 min with ER2 at 100° C. After blocking with 10% normal goat serum, the sections were incubated with primary mCD8a antibody (clone 4SM15, eBioscience, 2.5 μg/mL) for 60 min. Detection was carried out with anti-rat secondary antibody conjugated to HRP (GBI, D35-18) and visualized using DAB substrate.

CD8a staining was quantified using Definiens Tissue Studio software. ROIs were selected in regions of viable tissue; section edges and necrotic regions were excluded. The total number of cells was determined by counting hematoxylin-stained nuclei. Positive signal was detected by setting the threshold for DAB chromogen above background. Cells with positive staining of cytoplasmic/membrane regions were counted to obtain the total number of CD8a⁺ cells and divided by the total number of cells to obtain the percentage of CD8a⁺ cells.

RNA-Seq Analysis

RNAseq was performed with Qiagen targeted RNAseq panels consisting of 1278 total genes. EMT and fibrosis gene signatures were based on Qiagen gene lists and the VEGF pathway signature was based on the Biocarta VEGF pathway in Broad's Canonical Pathways. For these gene sets, signature scores are defined as the mean log₂ (fold-change) among all genes in each gene signature. These were calculated by adding a pseudocount of 0.5 TPM to all genes and samples, determining the log₂ (TPM), then subtracting the median log 2-TPM for each gene across all samples from the log₂-TPM for each gene. Signature scores for gene sets are shown as boxplots.

Statistical Analyses

Statistical analyses were performed using GraphPad Prism Software, version 7.0. For efficacy studies, tumor volume data are presented graphically as mean±SEM by symbols or as individual mice by lines. To assess differences in tumor volumes between treatment groups two-way analysis of variance (ANOVA) was performed followed by Tukey's multiple comparison test. A Kaplan-Meier plot was generated to show survival by treatment group and significance was assessed by log-rank (Mantel-Cox) test. For ex vivo lung imaging analysis, a Mann Whitney test was used to compare bioluminescence (photons/sec) between treatment groups. For quantification of the percentage of CD8a⁺ cell in IHC images, one-way ANOVA was performed followed by Dunnett's post-test to compare treatment groups to the triple combination therapy group. To compare signature scores across treatment groups, one-way ANOVA was performed followed by a Sidak's multiple comparison post-test.

The following embodiments are preferred:
1. A method for treating a cancer in a subject in need thereof, comprising administering to the subject a PD-1 axis binding antagonist, a TGFβ inhibitor and a DNA-PK inhibitor.
2. The method according to item 1, further comprising radiotherapy.
3. The method according to item 1 or 2, wherein the PD-1 axis binding antagonist and TGFβ inhibitor are fused.
4. The method according to any one of items 1 to 3, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.
5. The method according to item 4, wherein the PD-1 axis binding antagonist and TGFβ inhibitor are fused and the fusion molecule comprises the heavy chain having amino acid sequence of SEQ ID NO: 10 and the light chain having amino acid sequence of SEQ ID NO: 9.
6. The method according to any one of items 1 to 4, wherein the PD-1 axis binding antagonist is an anti-PD-L1 antibody and comprises the heavy chain having amino acid sequences of SEQ ID NOs: 7 or 8 and the light chain having amino acid sequence of SEQ ID NO: 9.
7. The method according to any one of items 1 to 4, wherein the PD-1 axis binding antagonist is avelumab.
8. The method according to any one of items 1 to 7, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof.
9. The method according to any one of items 1 to 8, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof,
   wherein the PD-1 axis binding antagonist and TGFβ inhibitor are fused, and
   wherein the fusion molecule comprises the heavy chain having amino acid sequence of SEQ ID NO: 10 and the light chain having amino acid sequence of SEQ ID NO: 9.
10. The method according to any one of items 1 to 9, wherein the subject is human.
11. The method according to any one of items 1 to 10, wherein the cancer is selected from cancer of the lung, head and neck, colon, neuroendocrine system, mesenchyme, breast, ovaries, pancreas, and histological subtypes thereof.
12. The method according to any one of items 1 to 11, wherein the cancer is selected from small-cell lung cancer (SCLC), non-small-cell lung cancer (NSCLC), squamous cell carcinoma of the head and neck (SCCHN), colorectal cancer (CRC), primary neuroendocrine tumors and sarcoma.
13. The method according to any one of items 1 to 12, wherein the PD-1 axis binding antagonist, TGFβ inhibitor and DNA-PK inhibitor are administered in a first-line treatment of the cancer.
14. The method according to any one of items 1 to 13, wherein the cancer is selected from the group of SCLC extensive disease (ED), NSCLC and SCCHN.
15. The method according to any one of items 1 to 14, wherein the subject underwent at least one round of prior cancer therapy.
16. The method according to item 15, wherein the cancer was resistant or became resistant to prior therapy.
17. The method according to any one of items 1 to 12, wherein the PD-1 axis binding antagonist, TGFβ inhibitor and DNA-PK inhibitor are administered in a second-line or higher treatment of the cancer.
18. The method according to item 17, wherein the cancer is selected from the group of pre-treated relapsing metastatic NSCLC, unresectable locally advanced NSCLC, pre-treated SCLC ED, SCLC unsuitable for systemic treatment, pre-treated relapsing or metastatic SCCHN, recurrent SCCHN eligible for re-irradiation, and pre-treated microsatellite status instable low (MSI-L) or microsatellite status stable (MSS) metastatic colorectal cancer (mCRC).
19. The method according to any one of items 1 to 18, wherein the PD-1 axis binding antagonist is an anti-PD-L1 antibody, and
   wherein the anti-PD-L1 antibody is administered via intravenous infusion over 50-80 minutes.
20. The method according to any one of items 1 to 19, wherein the PD-1 axis binding antagonist is an anti-PD-L1 antibody, and wherein the anti-PD-L1 antibody is administered once every two weeks (Q2W), at a dose of about 10 mg/kg body weight or about 800 mg.
21. The method according to any one of items 1 to 20, wherein the TGFβ inhibitor is administered via intravenous infusion.
22. The method according to any one of items 1 to 21, wherein the DNA-PK inhibitor is administered orally.
23. The method according to any one of items 1 to 22, wherein the DNA-PK inhibitor is administered once daily (QD) or twice daily (BID), at a dose of about 1 to about 800 mg.
24. The method according to item 23, wherein the DNA-PK inhibitor is administered twice daily (BID), at a dose of about 400 mg.
25. The method according to any one of items 1 to 24, further comprising administering a chemotherapy (CT), radiotherapy (RT), or chemotherapy and radiotherapy (CRT) to the subject.
26. The method according to item 25, wherein the chemotherapy is one or more selected from the group of etoposide, doxorubicin, topotecan, irinotecan, fluorouracil, a platin, an anthracycline, and a combination thereof.
27. The method according to item 26, wherein the chemotherapy is etoposide.
28. The method according to item 27, wherein etoposide is administered via intravenous infusion over about 1 hour.
29. The method according to item 27 or 28, wherein etoposide is administered on day 1 to 3 every three weeks (D1-3 Q3W), in an amount of about 100 mg/m².
30. The method according to item 26, wherein the chemotherapy is topotecan.
31. The method according to item 30, wherein topotecan is administered on day 1 to 5 every three weeks (D1-5 Q3W).
32. The method according to item 26, wherein the chemotherapy is cisplatin.
33. The method according to item 32, wherein cisplatin is administered via intravenous infusion over about 1 hour.
34. The method according to item 32 or 33, wherein cisplatin is administered once every three weeks (Q3W), in an amount of about at 75 mg/m².
35. The method according to item 26, wherein the chemotherapy is etoposide and cisplatin, and
   wherein both the etoposide and cisplatin are administered sequentially in either order or substantially simultaneously.
36. The method according to item 26, wherein the chemotherapy is anthracycline, and
   wherein the anthracycline is administered until reaching a maximal life-long accumulative dose.
37. The method according to item 25, further comprising radiotherapy,
   wherein the radiotherapy comprises about 35-70 Gy/20-35 fractions.
38. The method according to item 25 or 37, wherein the radiotherapy is selected from a treatment given with electrons, photons, protons, alfa-emitters, other ions, radio-nucleotides, boron capture neutrons and combinations thereof.
39. The method according to any one of items 1 to 38, which comprises a lead phase, optionally followed by a maintenance phase after completion of the lead phase.
40. The method according to item 39, wherein the PD-1 axis binding antagonist, TGFβ inhibitor and DNA-PK inhibitor are administered concurrently in either the lead or maintenance phase and optionally non-concurrently in the other phase, or the PD-1 axis binding antagonist, TGFβ inhibitor and DNA-PK inhibitor are administered non-concurrently in the lead and maintenance phase.
41. The method according to item 40, wherein the concurrent administration comprises the administration of the PD-1 axis binding antagonist, TGFβ inhibitor and DNA-PK inhibitor sequentially in either order or substantially simultaneously.
42. The method according to any one of items 39 to 41, wherein the lead phase comprises administration of the DNA-PK inhibitor alone or concurrently with one or more therapies selected from the group of the PD-1 axis binding antagonist, TGFβ inhibitor, chemotherapy and radiotherapy.
43. The method according to any one of items 39 to 42, wherein the maintenance phase comprises administration of the PD-1 axis binding antagonist alone or concurrently with the DNA-PK inhibitor or TGFβ inhibitor, or none of them.
44. The method according to any one of items 39 to 43, wherein the lead phase comprises the concurrent administration of the PD-1 axis binding antagonist, TGFβ inhibitor and DNA-PK inhibitor.
45. The method according to any one of items 39 to 43, wherein the lead phase comprises the administration of the DNA-PK inhibitor, and wherein the maintenance phase comprises the administration of the PD-1 axis binding antagonist and TGFβ inhibitor after completion of the lead phase.
46. The method according to item 39, wherein the lead phase comprises the concurrent administration of the DNA-PK inhibitor and etoposide, optionally together with cisplatin, wherein the maintenance phase comprises the administration of the PD-1 axis binding antagonist and TGFβ inhibitor, optionally together with the DNA-PK inhibitor, after completion of the lead phase, and wherein the cancer is SCLC ED.
47. The method according to any one of items 39 to 46, wherein the lead phase comprises the combination of the DNA-PK inhibitor, etoposide and cisplatin.
48. The method according to any one of items 39 to 47, wherein the lead phase comprises the concurrent administration of the PD-1 axis binding antagonist, TGFβ inhibitor, DNA-PK inhibitor and etoposide, optionally together with the cisplatin, and optionally further comprising the maintenance phase after completion of the lead phase, wherein the maintenance phase comprises the administration of the PD-1 axis binding antagonist and TGFβ inhibitor, and wherein the cancer is SCLC ED.
49. The method according to any one of items 39 to 48, wherein the lead phase comprises administration of the combination of the PD-1 axis binding antagonist, TGFβ inhibitor, DNA-PK inhibitor, etoposide and cisplatin.
50. The method according to item 26, wherein the chemotherapy is etoposide and the cancer is SCLC ED, and
   wherein etoposide is administered, optionally together with cisplatin, up to 6 cycles or until progression of SCLC ED.
51. The method according to any one of items 39 to 45, wherein the lead phase comprises the concurrent administration of the PD-1 axis binding antagonist, TGFβ inhibitor, DNA-PK inhibitor, irinotecan and fluorouracil, and wherein the cancer is mCRC MSI-L.
52. The method according to any one of items 39 to 45, wherein the lead phase comprises the concurrent administration of the PD-1 axis binding antagonist, TGFβ inhibitor, DNA-PK inhibitor and radiotherapy or chemoradiotherapy, wherein the maintenance phase comprises the administration of the PD-1 axis binding antagonist and TGFβ inhibitor after completion of the lead phase, and wherein the cancer is NSCLC or SCCHN.
53. The method according to any one of items 39 to 45, wherein the lead phase comprises the concurrent administration of the PD-1 axis binding antagonist, TGFβ inhibitor, DNA-PK inhibitor and radiotherapy, and wherein the cancer is NSCLC or SCCHN.
54. The method according to any one of items 1 to 53, wherein the cancer is selected based on PD-L1 expression in samples taken from the subject.
55. A pharmaceutical composition comprising a PD-1 axis binding antagonist, a TGFβ inhibitor, a DNA-PK inhibitor and at least a pharmaceutically acceptable excipient or adjuvant.
56. The pharmaceutical composition according to item 55, wherein the PD-1 axis binding antagonist and TGFβ inhibitor are fused.
57. The pharmaceutical composition according to item 55 or 56, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.
58. The pharmaceutical composition according to item 57, wherein the PD-1 axis binding antagonist and TGFβ inhibitor are fused and the fusion molecule comprises the heavy chain having amino acid sequence of SEQ ID NO: 10 and the light chain having amino acid sequence of SEQ ID NO: 9.
59. The pharmaceutical composition according to any one of items 55 to 57, wherein the PD-1 axis binding antagonist is an anti-PD-L1 antibody and comprises the heavy chain having amino acid sequences of SEQ ID NOs: 7 or 8 and the light chain having amino acid sequence of SEQ ID NO: 9.
60. The pharmaceutical composition according to any one of items 55 to 57, wherein the PD-1 axis binding antagonist is avelumab.
61. The pharmaceutical composition according to any one of items 55 to 60, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof.
62. The pharmaceutical composition according to any one of items 55 to 61, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof,
   wherein the PD-1 axis binding antagonist and TGFβ inhibitor are fused, and
   wherein the fusion molecule comprises the heavy chain having amino acid sequence of SEQ ID NO: 10 and the light chain having amino acid sequence of SEQ ID NO: 9.
63. The pharmaceutical composition according to any one of items 55 to 62 for use in therapy, preferably for use in treating cancer.
64. The pharmaceutical composition for use according to item 63, wherein the composition is used for treating cancer and the cancer is selected based on PD-L1 expression in samples taken from the subject.
65. A combination comprising a PD-1 axis binding antagonist, a TGFβ inhibitor and a DNA-PK inhibitor for use in therapy, preferably for use in treating cancer.
66. The combination for use according to item 65, wherein the PD-1 axis binding antagonist and TGFβ inhibitor are fused.
67. The combination for use according to item 65 or 66, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.
68. The combination for use according to any one of items 65 to 67, wherein the PD-1 axis binding antagonist and TGFβ inhibitor are fused and the fusion molecule comprises the heavy chain having amino acid sequence of SEQ ID NO: 10 and the light chain having amino acid sequence of SEQ ID NO: 9.
69. The combination for use according to any one of items 65 to 68, wherein the combination is used for treating cancer and the cancer is selected based on PD-L1 expression in samples taken from the subject to be treated.
70. A combination comprising a PD-1 axis binding antagonist, a TGFβ inhibitor and a DNA-PK inhibitor.
71. The combination according to item 70, wherein the PD-1 axis binding antagonist and TGFβ inhibitor are fused.
72. The combination according to item 70 or 71, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.
73. The combination according to any one of items 70 to 72, wherein the PD-1 axis binding antagonist and TGFβ inhibitor are fused and the fusion molecule comprises the heavy chain having amino acid sequence of SEQ ID NO: 10 and the light chain having amino acid sequence of SEQ ID NO: 9.
74. Use of combination for the manufacture of a medicament, preferably for the treatment of cancer, the combination comprising a PD-1 axis binding antagonist, a TGFβ inhibitor and a DNA-PK inhibitor.
75. The use according to item 74, wherein the PD-1 axis binding antagonist and TGFβ inhibitor are fused.
76. The use according to item 74 or 75, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.
77. The use according to any one of items 74 to 76, wherein the PD-1 axis binding antagonist and TGFβ inhibitor are fused and the fusion molecule comprises the heavy chain having amino acid sequence of SEQ ID NO: 10 and the light chain having amino acid sequence of SEQ ID NO: 9.
78. The use according to any one of items 74 to 77, wherein the combination is used for the manufacture of a medicament for the treatment of cancer, and wherein the cancer is selected based on PD-L1 expression in samples taken from the subject.
79. A kit comprising a PD-1 axis binding antagonist and a package insert comprising instructions for using the PD-1 axis binding antagonist in combination with a TGFβ inhibitor and a DNA-PK inhibitor to treat or delay progression of a cancer in a subject.
80. A kit comprising a DNA-PK inhibitor and a package insert comprising instructions for using the DNA-PK inhibitor in combination with a PD-1 axis binding antagonist and a TGFβ inhibitor to treat or delay progression of a cancer in a subject.
81. A kit comprising a TGFβ inhibitor and a package insert comprising instructions for using the TGFβ inhibitor in combination with a PD-1 axis binding antagonist and a DNA-PK inhibitor to treat or delay progression of a cancer in a subject.
82. A kit comprising a PD-1 axis binding antagonist and a TGFβ inhibitor and a package insert comprising instructions for using the PD-1 axis binding antagonist and TGFβ inhibitor in combination with a DNA-PK inhibitor to treat or delay progression of a cancer in a subject.
83. The kit according to item 82, wherein the PD-1 axis binding antagonist and TGFβ inhibitor are fused.
84. A kit comprising a PD-1 axis binding antagonist and a DNA-PK inhibitor and a package insert comprising instructions for using the PD-1 axis binding antagonist and DNA-PK inhibitor in combination with a TGFβ inhibitor to treat or delay progression of a cancer in a subject.
85. A kit comprising a TGFβ inhibitor and a DNA-PK inhibitor and a package insert comprising instructions for using the TGFβ inhibitor and DNA-PK inhibitor in combination with a PD-1 axis binding antagonist to treat or delay progression of a cancer in a subject.
86. A kit comprising a PD-1 axis binding antagonist, a TGFβ inhibitor and a DNA-PK inhibitor and a package insert comprising instructions for using the PD-1 axis binding antagonist, TGFβ inhibitor and DNA-PK inhibitor to treat or delay progression of a cancer in a subject.
87. The kit according to any one of items 79 to 86, wherein the instructions state that the medicaments are intended for use in treating a subject having a cancer that tests positive for PD-L1 expression by an immunohistochemical assay.
88. A method for advertising a PD-1 axis binding antagonist, a TGFβ inhibitor and a DNA-PK inhibitor comprising promoting, to a target audience, the use of the combination for treating a subject with a cancer, preferably a cancer selected based on PD-L1 expression in samples taken from the subject.
89. The method according to item 88, wherein the PD-L1 expression is determined by immunohistochemistry using one or more primary anti-PD-L1 antibodies.
90. The method according to any one of items 1 to 18, wherein the PD-1 axis binding antagonist and the TGFβ inhibitor are fused as the anti-PD-L1/TGFβ Trap molecule; and wherein the anti-PD-L1/TGFβ Trap molecule is administered at a dose of 1200 mg IV every two weeks, a dose of 1800 mg IV every three weeks or a dose of 2400 mg IV every three weeks.

What is claimed is:

1-12. (canceled)

13. A pharmaceutical composition comprising a PD-1 axis binding antagonist, a TGFβ inhibitor, a DNA-PK inhibitor and at least one pharmaceutically acceptable excipient or adjuvant.

14. A kit comprising a PD-1 axis binding antagonist, a TGFβ inhibitor and a DNA-PK inhibitor.

15. A kit comprising a PD-1 axis binding antagonist and a package insert comprising instructions for using the PD-1 axis binding antagonist in combination with a TGFβ inhibitor and a DNA-PK inhibitor to treat or delay progression of a cancer in a subject.

16. A kit comprising a PD-1 axis binding antagonist, a TGFβ inhibitor and a package insert comprising instructions for using the PD-1 axis binding antagonist and TGFβ inhibitor in combination with a DNA-PK inhibitor to treat or delay progression of a cancer in a subject.

17. A method of treating a disease in a subject the method comprising the step of administering a PD-1 axis binding antagonist, a TGFβ inhibitor and a DNA-PK inhibitor to the subject.

18. The method of claim 17, wherein the disease is cancer.

19. The method of claim 18, further comprising administering chemotherapy, radiotherapy or chemoradiotherapy to the subject.

20. The method of claim 18, wherein the PD-1 axis binding antagonist is a is an anti-PD-1 or anti-PD-L1 antibody.

21. The method of claim 20, wherein the PD-1 axis binding antagonist comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

22. The method of claim 18, wherein the TGFβ inhibitor is a polypeptide comprising a human TGFβRII, or a fragment capable of binding TGFβ.

23. The method of claim 18, wherein the PD-1 axis binding antagonist and TGFβ inhibitor are fused to form a fusion molecule.

24. The method of claim 23, wherein the fusion molecule comprises the heavy chain having amino acid sequence of SEQ ID NO: 10 and the light chain having amino acid sequence of SEQ ID NO: 9.

25. The method of claim 18, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof.

26. The method of claim 18, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof, wherein the PD-1 axis binding antagonist is an anti-PD-1 or anti-PD-L1 antibody, and wherein the TGFβ inhibitor is a polypeptide comprising a human TGFβRII, or a fragment capable of binding TGFβ.

27. The method of claim 26, wherein the PD-1 axis binding antagonist and TGFβ inhibitor are fused.

28. The method of claim 27, wherein the fusion molecule comprises the heavy chain having amino acid sequence of SEQ ID NO: 10 and the light chain having amino acid sequence of SEQ ID NO: 9.

29. The method of claim 18, wherein before administering into the subject the PD-1 axis binding antagonist, a TGFβ inhibitor, and a DNA-PK inhibitor, a sample taken from the subject has been determined to show PD-L1 expression.

30. The method of claim 29 further comprising administering radiotherapy to the subject.


SEQUENCE LISTING (13 sequences)

SEQ ID NO: 1 - protein, 5 aa, Artificial Sequence
  Synthetic peptide
SYIMM

SEQ ID NO: 2 - protein, 17 aa, Artificial Sequence
  Synthetic peptide
SIYPSGGITFYADTVKG

SEQ ID NO: 3 - protein, 11 aa, Artificial Sequence
  Synthetic peptide
IKLGTVTTVDY

SEQ ID NO: 4 - protein, 14 aa, Artificial Sequence
  Synthetic peptide
TGTSSDVGGYNYVS

SEQ ID NO: 5 - protein, 7 aa, Artificial Sequence
  Synthetic peptide
DVSNRPS

SEQ ID NO: 6 - protein, 10 aa, Artificial Sequence
  Synthetic peptide
SSYTSSSTRV

SEQ ID NO: 7 - protein, 450 aa, Artificial Sequence
  Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Artificial Sequence
  Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 216 aa, Artificial Sequence
  Synthetic peptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 10 - protein, 607 aa, Artificial Sequence
  Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 12 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 13 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD